(19) United States
(12) Patent Application Publication
(10) Pub. No.: US 2023/0372654 A1
(43) Pub. Date: Nov. 23, 2023

(54) BREATHING MASK WITH PARTICLE FILTER

(71) Applicant: Loewenstein Medical Technology S.A., Luxembourg (DE)

(72) Inventor: Martin EIFLER, Gluechstadt (DE)

(21) Appl. No.: 18/318,089

(22) Filed: May 16, 2023

(30) Foreign Application Priority Data
May 17, 2022 (DE) 102022112288.3

(51) Int. Cl. A61M 16/06; A61M 16/10

(52) U.S. Cl. CPC A61M 16/0605; A61M 16/105

(57) ABSTRACT

A breathing mask comprises a mask body, a mask bead, at least one mask wing, and at least one filter element. The mask bead is connected to the mask body, and the mask wing is connected to the mask body and/or the mask bead. The mask bead is formed at least in sections for contact on the skin of a user and presses against the skin of the user during use of the breathing mask in such a way that the breathing mask terminates essentially respiratory gas-tight. The mask bead includes a receptacle opening, which is configured to receive at least nose and mouth of the user during use of the breathing mask. The filter element is connected to the mask wing and is configured in such a way that respiratory gas can flow through it at least in some areas.

### CROSS-REFERENCE TO RELATED APPLICATIONS

The present application claims priority under 35 U.S.C. § 119 of German Patent Application No. 102022112288.3, filed Jun. 17, 2022, the entire disclosure of which is expressly incorporated by reference herein.

### BACKGROUND OF THE INVENTION

### 1. Field of the Invention

The present invention relates to a breathing mask with particle filter.

### 2. Discussion of Background Information

Particle-filtering half masks are used as respiratory protection against aerosols made up of solid or liquid, not highly volatile particles. They are tested according to European Standard DIN EN 149 and meet the requirements of this standard. The standard distinguishes the device classes FFP1, FFP2, and FFP3 depending on the retention capacity of the particle filter. The mask typically consists completely of the non-replaceable filter material. Examples of such masks are disclosed in DE 10 2014 221 311 B3 and DE 20 2013 011 420 U1, the entire disclosures of which are expressly incorporated by reference herein.

It is necessary to draw a distinction between the above and “half masks or full masks with a particle filter”, which have one or more attachment options for replaceable particle filters. Half masks or full masks with replaceable particle filters according to DE 10 2014 001 937 B3 or DE 20 2014 001 315 U1, the entire disclosures of which are expressly incorporated by reference herein, are presently quite bulky masks, which are used in particular in the industrial sector and greatly restrict the field of view. Moreover, such masks have at least one valve for inhalation and/or exhalation.

The leak-tightness is decisive for the protective effect of a breathing mask. This results from the filter penetration and the so-called fitting leakage, which arises due to leaks between the sealing line of the mask and the face of the wearer. The protective effect increases from an FFP1 mask (total leakage maximum 22%) via an FFP2 mask (total leakage maximum 8%) to the FFP3 mask (total leakage maximum 2%). The breathing resistance of the mask also rises with the increase in the protective effect. The exhalation resistance is reduced by an exhalation valve. The particle-filtering half mask is thus less burdensome for the wearer and is therefore preferably to be used.

Medical breathing masks, which are to be used for protection from infection, also require particle-filtering properties in order to offer protection from infectious particles and/or aerosols. Viruses, bacteria, and other infectious material can be bound to ultrasmall droplets and thus captured in the filter. Infection protection masks can offer protection against bacteria and viruses for both the wearer and other persons.

Breathing masks according to the prior art have the following significant disadvantages:
large amount of waste in the case of disposable masks
little adjustability and high leakage rates with uniform sizes or uniform shapes
clumsy construction
breathing masks having exhalation valves can protect the user but not other persons, since infectious material is emitted unfiltered into the surroundings via the exhalation valve
costly production
complicated handling, in particular upon filter change

In view of the foregoing, it would be advantageous to have available a breathing mask which is adjustable to all facial shapes and is simple to handle and nonetheless offers secure protection and does not impair the user.

### SUMMARY OF THE INVENTION

The invention provides a breathing mask comprising a mask body, a mask bead, at least one mask wing, and at least one filter element. The mask bead is connected to the mask body, and the mask wing is connected to the mask body and/or the mask bead. The mask bead is designed at least in sections for contact on the skin of a user, and the mask bead presses against the skin of the user during use of the breathing mask in such a way that the breathing mask is terminated essentially respiratory gas tight. The mask bead comprises a receptacle opening, which is designed and configured to receive at least the nose and mouth of the user during use of the breathing mask. The filter element is connected to the mask wing, and is configured and designed in such a way that respiratory gas can flow through it at least in some areas thereof.

In some embodiments, the breathing mask is characterized by the fact that the breathing mask comprises at least two mask wings, which are each connected to at least one filter element. In some embodiments, the breathing mask is characterized by the fact that the connection of mask wings and filter elements is reversible or irreversible.

In some embodiments, the breathing mask is characterized by the fact that the breathing mask is in a usage state when the filter elements are connected to the mask wings.

In some embodiments, the breathing mask is characterized by the fact that the filter element is a filter material and/or comprises a filter material.

In some embodiments, the breathing mask is characterized by the fact that the filter material filters and holds back inorganic particles and/or organic particles, in particular germs from the respiratory gas. In some embodiments, the breathing mask is characterized by the fact that the filter material is manufactured from polymer fibers, for example made of polypropylene (PP) and/or polytetrafluoroethylene (PTFE).

In some embodiments, the breathing mask is characterized by the fact that the filter material has an area of at least 5 cm², wherein the area of the filter material is preferably in a range between 35 cm² and 55 cm².

In some embodiments, the breathing mask is characterized by the fact that the filter material is exchangeable.

In some embodiments, the breathing mask is characterized by the fact that the mask body, the mask bead, and the mask wings having the filter elements define a respiratory gas space during use of the breathing mask, in which the mouth and nose of the user are located, wherein a respiratory gas flow away from the mouth and nose of the user and toward the mouth and nose of the user is exclusively possible through the filter material.

In some embodiments, the breathing mask is characterized by the fact that the respiratory gas space has a volume which is sufficiently small that CO₂ is washed out from the respiratory air of the user. In some embodiments, the breathing mask is characterized by the fact that the respiratory gas space has a volume of at most about 500 ml, preferably at most about 200 ml, for example at most about 120 ml.

In some embodiments, the breathing mask is characterized by the fact that the breathing mask is manufactured from an elastomeric and/or a dimensionally stable plastic. In some embodiments, the breathing mask is characterized by the fact that the breathing mask is manufactured from a polymer selected from polycarbonates (PC), polystyrenes (PS), polyamides (PA), polypropylenes (PP), polyoxymethylenes (POM), polyacrylonitriles (PAN), thermoplastic elastomers (TPE), acrylonitrile-styrene acrylates (ASA), acrylonitrile-butadiene-styrenes (ABS), styrene-acrylonitriles (SAN), styrene-methylmethacrylates (SMMA), polyethylenes (PE), cycloolefin copolymers (COC), polytetrafluorethylenes (PTFE), silicones.

In some embodiments, the breathing mask is characterized by the fact that the mask body and/or the mask bead and/or the mask wings are integrally manufactured. In some embodiments, the breathing mask is characterized by the fact that at least the mask body and the mask wings are integrally manufactured and the filter elements are reversibly connected to the mask wings.

In some embodiments, the breathing mask is characterized by the fact that the filter elements are manufactured from a dimensionally stable polymer, for example from polypropylene.

In some embodiments, the breathing mask is characterized by the fact that the mask body, the mask bead, and the mask wings are essentially manufactured from an elastomeric polymer. In some embodiments, the breathing mask is characterized by the fact that the mask body, the mask bead, and the mask wings are essentially manufactured from a silicone. In some embodiments, the breathing mask is characterized by the fact that the silicone is transparent, wherein the silicone has a light transmission of at least about 50%, preferably of at least about 80%, particularly preferably of at least about 90%.

In some embodiments, the breathing mask is characterized by the fact that the mask body, the mask bead, and the mask wings have a Shore hardness of from about 30 Shore A to about 60 Shore A. In some embodiments, the breathing mask is characterized by the fact that the mask body, the mask bead, and the mask wings have a maximum extensibility from about 100% to about 800%.

In some embodiments, the breathing mask is characterized by the fact that further materials for reinforcing and/or stiffening are introduced and/or applied in and/or on the material of the mask body.

In some embodiments, the breathing mask is characterized by the fact that the breathing mask can be disinfected and/or sterilized. In some embodiments, the breathing mask is characterized by the fact that at least the mask body, the mask bead, and/or the mask wings can be autoclaved.

In some embodiments, the breathing mask is characterized by the fact that the breathing mask comprises at least one strap bracket, which is configured to receive at least one head fastening strap. In some embodiments, the breathing mask is characterized by the fact that the strap brackets are formed as a projection and contain at least one feedthrough opening to feed through the head fastening strap. In some embodiments, the breathing mask is characterized by the fact that the strap brackets are arranged on the mask wings and/or on the filter elements. In some embodiments, the breathing mask is characterized by the fact that the strap brackets are arranged on the filter elements.

In some embodiments, the breathing mask is characterized by the fact that the mask wings comprise at least one wing duct having a lumen.

In some embodiments, the breathing mask is characterized by the fact that the mask wings comprise at least one receptacle device for receiving the filter element, wherein the receptacle device is formed substantially complementary in shape to the filter element.

In some embodiments, the breathing mask is characterized by the fact that the receptacle device and the filter element essentially have a polygonal basic shape, preferably a square basic shape. In some embodiments, the breathing mask is characterized by the fact that the basic shape of receptacle device and filter element is essentially rhombic or trapezoidal, preferably trapezoidal.

In some embodiments, the breathing mask is characterized by the fact that the receptacle device comprises at least one receptacle side wall, which is elastically deformable. In some embodiments, the breathing mask is characterized by the fact that the filter element is at least partially enclosed by the receptacle side wall after being received in the receptacle device and is fixed by this wall in the receptacle device.

In some embodiments, the breathing mask is characterized by the fact that the filter element comprises a front part and a rear part and the filter material, wherein the filter material is arranged between the front part and the rear part. In some embodiments, the breathing mask is characterized by the fact that the front part and the rear part are at least partially irreversibly connected to one another or are reversibly connected to one another.

In some embodiments, the breathing mask is characterized by the fact that the filter material is arranged exchangeably between the front part and the rear part.

In some embodiments, the breathing mask is characterized by the fact that the filter element comprises at least one duct having a lumen. In some embodiments, the breathing mask is characterized by the fact that the rear part comprises a rear wall and a side wall, which delimit the duct at least in some areas.

In some embodiments, the breathing mask is characterized by the fact that the rear part comprises at least one duct wall having a base and an end face, wherein the duct wall divides the duct into subunits.

In some embodiments, the breathing mask is characterized by the fact that the rear part comprises a support surface, wherein the filter material is placed on the support surface and/or on the end faces of the duct walls in such a way that the duct is delimited at least in some areas by the filter material.

In some embodiments, the breathing mask is characterized by the fact that the front part comprises an opening, through which an air exchange takes place between the respiratory gas space and the ambient air. In some embodiments, the breathing mask is characterized by the fact that the filter material is placed between the respiratory gas space and the opening in such a way that the air exchange exclusively takes place through the filter material.

In some embodiments, the breathing mask is characterized by the fact that the filter element includes a tunnel element having a lumen. In some embodiments, the breathing mask is characterized by the fact that the rear part comprises the tunnel element. In some embodiments, the breathing mask is characterized by the fact that the tunnel element is at least partially arranged in the wing duct of the mask wings in a usage state of the breathing mask.

In some embodiments, the breathing mask is characterized by the fact that the tunnel element includes at least one locking projection, via which the filter element is locked in the mask wings. In some embodiments, the breathing mask is characterized by the fact that the rear part and/or the front part comprises at least one locking rail, via which the filter element is locked in the mask wings.

In some embodiments, the breathing mask is characterized by the fact that the wing duct, the tunnel element, and the duct are connected to one another in a gas-conducting manner.

In some embodiments, the breathing mask is characterized by the fact that the lumina at least partially define the respiratory gas space, wherein the breathing mask is configured and designed in such a way that a substantially interference-free flow is enabled through the respiratory gas space and the filter material.

In some embodiments, the breathing mask is characterized by the fact that the respiratory gas flows from the mouth and/or nose of the user via the lumen of the wing duct into the lumen of the tunnel element and from the lumen of the tunnel element into the lumen of the duct and from the lumen of the duct exclusively through the filter material and vice versa.

In some embodiments, the breathing mask is characterized by the fact that the wing duct and/or the tunnel element have a hydraulic diameter of at least about 10 mm, preferably at least about 12 mm, particularly preferably at least about 15 mm.

The present invention further provides a filter element as set forth in the claims for a breathing mask according to the invention.

### BRIEF DESCRIPTION OF THE DRAWINGS

Exemplary embodiments of the breathing mask according to the invention and the filter element according to the invention are shown in the drawings. In the drawings:

FIG. 1 shows an illustration of a breathing mask from the front, wherein FIG. 1A shows the breathing mask with filter elements and FIG. 1B shows the breathing mask without filter elements.

FIG. 2 shows a perspective illustration of a breathing mask, wherein FIG. 2A shows the breathing mask with filter elements and FIG. 2B shows the breathing mask without filter elements.

FIG. 3 shows an illustration of a breathing mask having filter elements from the rear.

FIGS. 4A-C show illustrations of an exemplary mask bead of the breathing mask from different perspectives.

FIGS. 5A and 5C show an illustration of a breathing mask with filter elements from above.

FIG. 5B shows a breathing mask with filter elements from below.

FIG. 6 shows an illustration of a breathing mask with filter elements from the side.

FIG. 7 shows a simplified view of a breathing mask in cross-section upon contact on the head of a user.

FIG. 8 shows an exploded view of a filter element.

FIGS. 9A-E show a rear part of a filter element from different perspectives.

FIGS. 10A-E show a front part of a filter element from different perspectives.

FIGS. 11A-E show a filter element in a usage state from different perspectives.

FIG. 12 shows a perspective illustration of a breathing mask with filter elements in cross-section and

FIG. 12A shows a detail view from FIG. 12.

FIG. 13 shows an illustration of a breathing mask in cross-section.

The coordinate systems illustrated in some drawings are used to clarify the alignment of the views. The x, y, and z axes—if not described otherwise—describe the same direction in each figure. A direction of a figure designated with a x axis thus generally corresponds to the direction of the x axis of the other figures.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

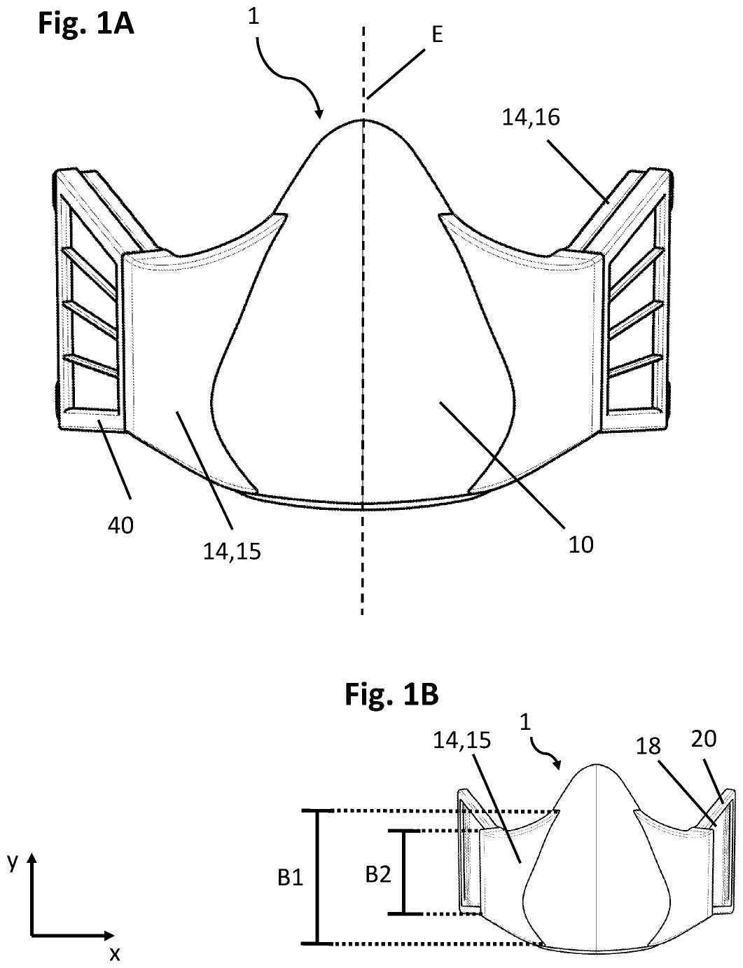

FIG. 1 shows an illustration of a breathing mask 1 from the front, wherein FIG. 1A shows the breathing mask 1 with filter elements 40 and FIG. 1B shows the breathing mask 1 without filter elements 40. It is apparent from FIG. 1 that the breathing mask 1 is designed to be essentially mirror symmetrical via a plane of symmetry E. A left half and a right half can be defined via the plane of symmetry E, which can preferably be designed symmetrically.

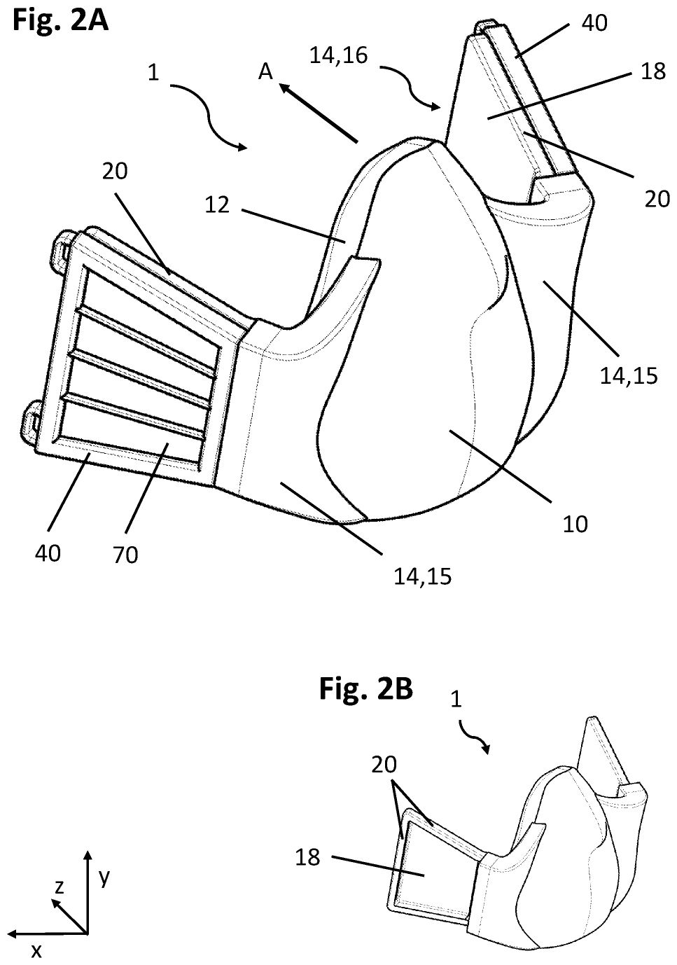

FIG. 2 shows a perspective illustration of a breathing mask 1, wherein FIG. 2A shows the breathing mask 1 with filter elements 40 and FIG. 2B shows the breathing mask 1 without filter elements 40. The breathing mask 1 comprises a mask body 10, a mask bead 12, and at least one mask wing 14. The mask wings 14 each comprise at least one receptacle device 16 and at least one wing duct 15. The breathing mask 1 moreover comprises at least one filter element 40, which can be connected reversibly and/or irreversibly via the receptacle device 16 of the mask wings 14 to the breathing mask 1. The filter element 40 is preferably reversibly connected to the breathing mask 1.

In one exemplary embodiment according to the drawings shown, the breathing mask 1 comprises two mask wings 14 each having a receptacle device 16, which can each receive a filter element 40. In some embodiments, it is also conceivable that the breathing mask 1 includes more than two mask wings 14, for example four or more (not shown).

The breathing mask 1 is in a usage state when the at least one filter element 40 is connected to the breathing mask 1. The breathing mask 1 can then be applied to the head 90 by a user (not shown) and the user can breathe. During breathing with applied breathing mask 1, the inhalation and exhalation air is essentially conducted through the filter elements 40. The general direction toward the user is indicated by the arrow designated by A in FIG. 2A. During use, the mask bead 12 thus points in the direction toward the user.

The filter element 40 can be a filter material 70 in a simple embodiment. In one preferred embodiment, the filter element 40 can comprise a filter material 70. In a specific exemplary embodiment according to the FIG.s, a particle-filtering filter material 70 can be introduced into the filter element 40 to produce a particle-filtering usage state. The material of the filter material 70 is to be selected so that organic and/or inorganic particles are filtered and held back by the filter material 70 from the respiratory gas or the respiratory air. The filter material 70 is configured and designed to filter and hold back germs from the respiratory gas or the respiratory air. Germs in the meaning of the invention comprise in particular microorganisms and/or ultrasmall living beings such as viruses, viroids, bacteria, prions, protists, algae, parasites, or fungi, and the derivatives or precursors thereof such as fungus spores, spores, allergens, toxins, and the like. Germs can be understood in particular as pathogenic agents and/or pathogens.

For example, the filter material 70 consists of polypropylene (PP) and/or polytetrafluoroethylene (PTFE). For example, however, other materials are also conceivable for use as the filter material 70, for example other polmer fibers arranged in a fine mesh. The filter material 70 can be designed as a disposable filter. It is also conceivable that the filter material 70 can be reprocessed after use and can be used multiple times.

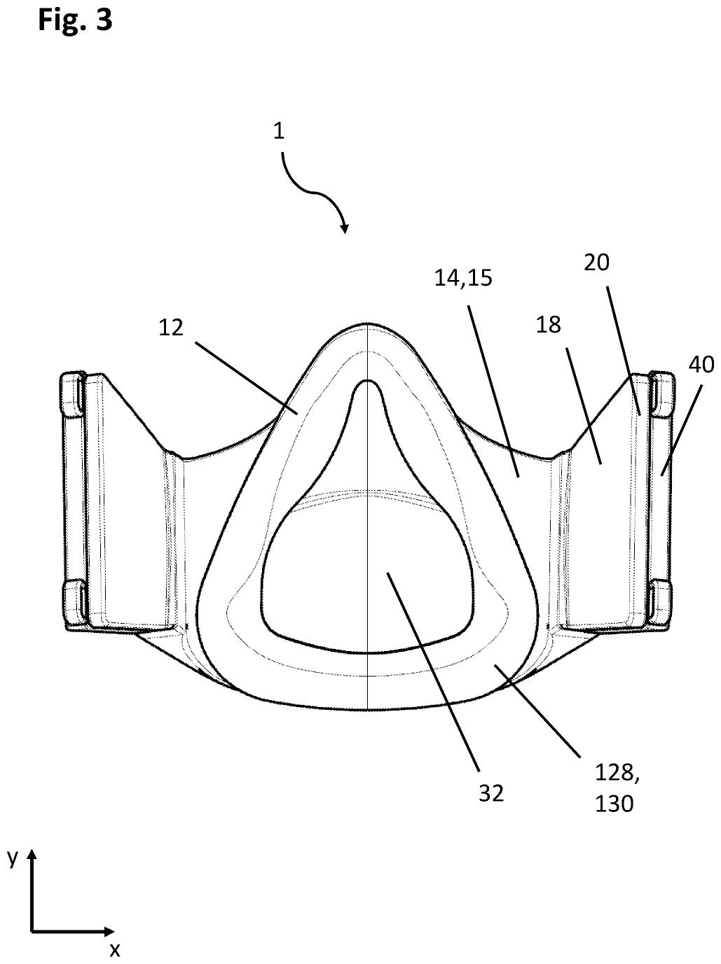

FIG. 3 shows an illustration of a breathing mask 1 having filter elements 40 from the rear. FIG. 3 shows the mask bead 12. The mask bead 12 includes a contact area 128 embodied as a sealing lip 130 and is configured in such a way that the breathing mask 1 presses at least in sections against the facial skin with the sealing lip 130 during use and terminates essentially respiratory gas-tight. The mask bead 12 at least partially delimits a receptacle opening 32. The receptacle opening 32 is configured to receive at least the nose and mouth of the user during use of the breathing mask 1. The mask bead 12 then presses against the face of the user in a respiratory gas-tight manner and the breathing mask 1 as a whole encloses at least the nose and mouth of the user. In some embodiments, the breathing mask 1 can partially or completely also enclose the chin of the user in addition to the mouth and nose during use.

In one exemplary embodiment, at least mask body 10 and mask wing 14 are embodied integrally. Mask bead 12 and mask body 10 can also be embodied integrally. In one exemplary specific embodiment, mask body 10, mask bead 12, and mask wing 14 can be produced in a production method and embodied integrally. The integral nature can also be achieved in that one or more parts are produced individually and subsequently irreversibly connected to one another. An irreversible connection can be achieved, for example, by adhesive bonding, extrusion coating, welding, or other connection methods.

A two-piece or multipiece embodiment is also possible. For example, the mask bead 12 can in some embodiments be manufactured in a separate production step and reversibly plugged onto the mask body 10 (not shown).

The filter element 40 is reversibly connected to the breathing mask 1 in one preferred embodiment. For this purpose, the filter element 40 can be connected to the receptacle device 16 of the mask wing 14 (see, for example FIGS. 1A, 2A).

The above-mentioned components of the breathing mask 1, namely mask body 10, mask bead 12, and mask wing 14, receptacle device 16, and filter element 40, are preferably produced from a polymer. The polymer can be elastic or elastomeric and/or dimensionally stable.

Suitable polymers can be selected from polycarbonates (PC), polystyrenes (PS), polyamides (PA), polypropylenes (PP), polyoxymethylenes (POM), polyacrylonitriles (PAN), thermoplastic elastomers (TPE), acrylonitrile-styrene acrylates (ASA), acrylonitrile-butadiene-styrenes (ABS), styrene-acrylonitriles (SAN), styrene-methylmethacrylates (SMMA), polyethylenes (PE), cycloolefin copolymers (COC), polytetrafluorethylene (PTFE), silicones. When selecting suitable materials, it is to be ensured that they are not permeable to respiratory gas—with the exception of the filter element 40 or with the exception of individual components of the filter element 40, such as the filter material 70.

The components of the breathing mask 1 can be produced from the same material or from different materials. In one specific exemplary embodiment, mask body 10, mask bead 12, and mask wing 14 including receptacle device 16 can be produced from an elastomeric plastic. For example, mask body 10, mask bead 12, and mask wing 14 including receptacle device 16 are made of silicone. The filter elements 40 can be essentially produced from a dimensionally stable plastic. For example, the filter elements 40 are essentially made of polypropylene.

Due to the selection of an elastomeric polymer, the mask body 10 as such can be dimensionally stable, but can be elastically deformable upon tensile and/or compressive load. In addition to the base material of the individual components, further materials can be used. For example, it is conceivable that further materials for reinforcing and/or stiffening are introduced and/or applied in the base material.

The base material of mask body 10, mask bead 12, and mask wing 14 including receptacle device 16 can be silicone, for example. Mask body 10, mask bead 12, mask wing 14, and receptacle device 16 can thus be elastically deformable. The Shore hardness of the material used is, for example, from about 30 to about 60 Shore A, wherein a maximum extensibility of from about 100% to about 800% can be achieved.

An integral embodiment of the breathing mask 1, in particular from silicone, offers the advantage that the breathing mask 1 can be cleaned and disinfected and/or prepared in another way easily, safely, and multiple times without the breathing mask 1 experiencing quality losses. Silicone has good chemical and temperature resistance and is accordingly well suitable for frequent cleaning or disinfection. In particular, the breathing mask 1 without filter elements 40 can be autoclaved. Moreover, silicone is biologically well compatible.

The breathing mask 1 and in particular the mask body 10 is preferably manufactured from a transparent or translucent material, for example a transparent silicone. The material of the mask body 10 preferably has a light transmission of at least about 50%, preferably at least about 80%. In a specific exemplary embodiment, the light transmission is at least about 90%.

The use of a transparent material for the breathing mask 1 and the arrangement of the filter element 40 in the area of the mask wings 14 offers the advantage that in particular the mouth of the user is visible during use of the breathing mask 1. This can positively influence the visual effect of the breathing mask 1, since facial expressions of the user can be perceived better from the surroundings. Moreover, the breathing mask 1 offers the advantage that the mouth is visible when speaking, which can increase comprehensibility of the speech.

The use of an elastically deformable material for the breathing mask 1 offers the advantage that the breathing mask 1 as a whole—with the exception of the filter element 40—is soft. The breathing mask 1 can thus bend around the chin and can thus preferably adapt itself to any facial shape. The breathing mask 1 can deform in the section spanning the nose area in the case of high nose bridges and is thus suitable for various nose shapes. A bendable, stiffening element such as a wire or the like can optionally be arranged in the section spanning the nose area. Better adaptation to the nose can be ensured via this element.

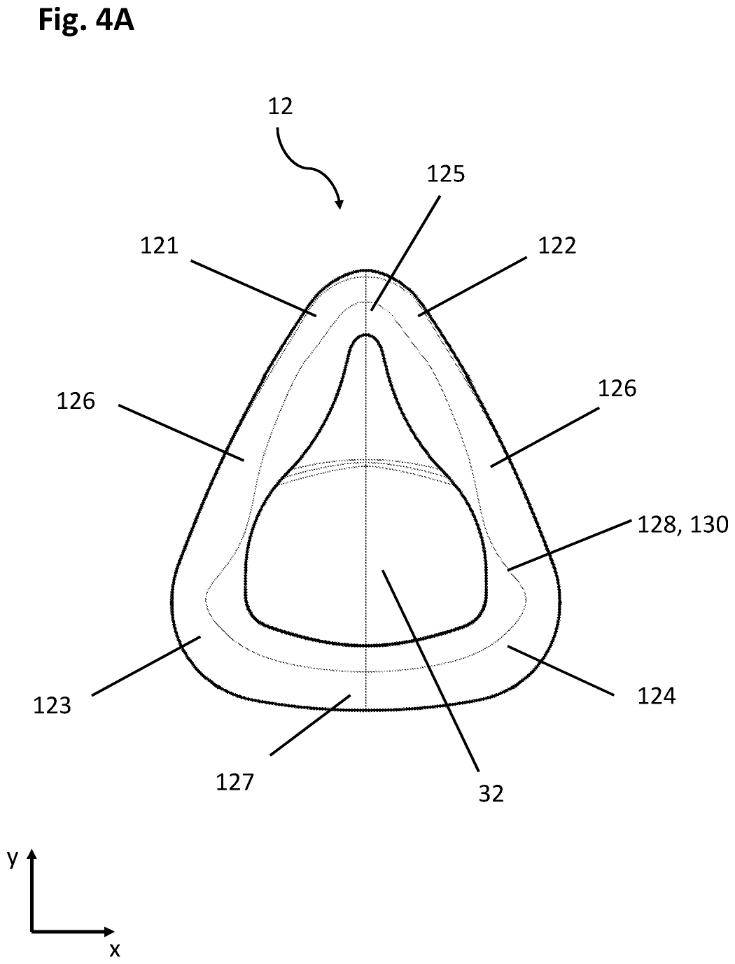

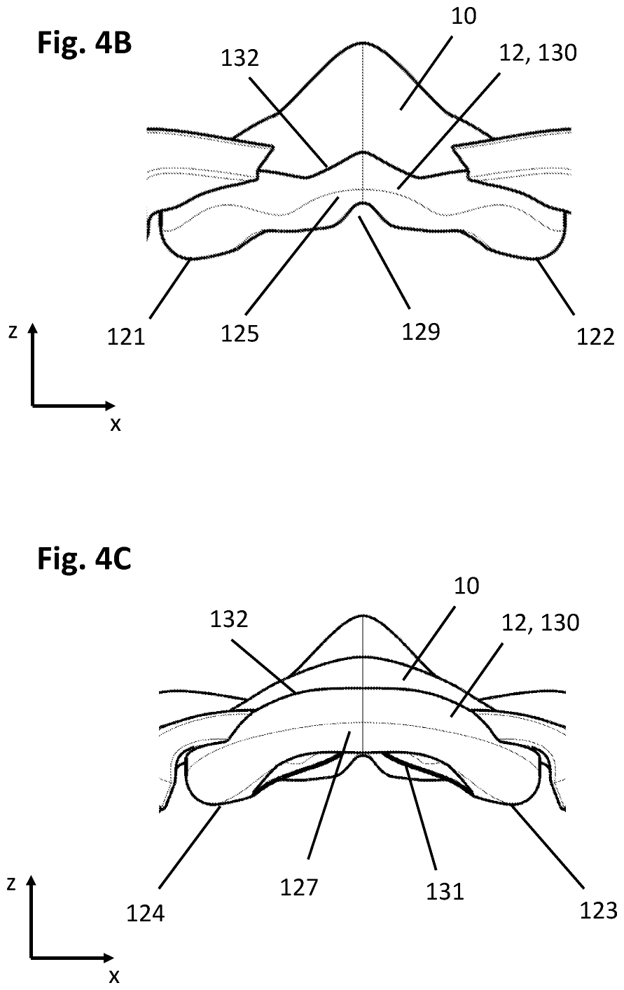

FIGS. 4A, 4B, and 4C show illustrations of an exemplary mask bead 12 of the breathing mask 1 from different perspectives, wherein FIG. 4A shows the mask bead 12 in a frontal view, thus the side facing toward the user, FIG. 4B shows the mask bead from above, and FIG. 4C shows the mask bead from below.

The mask bead 12 can be divided into two lateral areas 126, a base area 127, and a nose bridge area 125. The mask bead 12 delimits the receptacle opening 32 to the sides using the lateral areas 126, the base area 127, and the nose bridge area 125. The receptacle opening 32 is designed to enclose at least the nose and mouth of the user. In some embodiments, the receptacle opening 32 can additionally be designed to at least partially receive and/or enclose the chin of the user.

The mask bead 12 can be embodied as a sealing lip 130 and can include a contact area 128 (see FIG. 4A) and an edge 131 (see FIG. 4C). The contact area 128 represents the side facing toward the user. The mask bead 12 presses against the face of the user via the contact area 128 of the sealing lip 130 and terminates respiratory gas-tight. The sealing lip 130 protrudes in an arc into the receptacle opening 32, for example, starting from the lateral areas 126, the base area 127, and the nose bridge area 125 (see FIGS. 4B/4C). The contact area 128 simultaneously represents the front side, i.e., the side facing toward the user, and the delimitation of the receptacle opening 32 by the mask bead 12 here.

In the mask bead 12 shown by way of example in FIGS. 4A-C, corner points 121, 122, 123, and 124 are additionally formed, which are solidified in relation to the remaining mask bead 12 or have an elevated wall thickness, for example. These corner points 121, 122, 123, 124 are arranged, for example, so that they rest on less sensitive points in the face of the user and thus additionally support the mask bead 12. Due to the supporting function of the corner points 121, 122, for example, other areas of the mask bead 12 can also be embodied having a lesser wall thickness. For example, the mask bead 12 can have a significantly lower wall thickness in the nose bridge area 125. This can have the result that in this area the mask bead 12 is not supported in relation to the face and is configured more flexibly. The mask bead 12 can thus adapt itself more easily in this area to the facial contour without exerting an additional pressure in the sensitive area of the nose bridge of the user.

Alternatively or additionally to the low wall thickness, a notch 129 can be arranged in the nose bridge area 125 (FIG. 4B), which additionally enables easier adaptation to the facial contours of the user.

A transition 132 can be formed between mask body 10 and mask bead 12. In one exemplary embodiment according to the drawings, mask bead 12 and mask body 10 can be integrally manufactured. In the case of integral manufacturing, the transition 132 cannot extend noticeably. However, in some embodiments a change in the material quality and/or material thickness can also be introduced at the transition 132.

The mask bead 12 can in some embodiments also be provided separately and can be connected to the mask body 10 for use. In the case of separate manufacturing of the mask bead 12, for example if the mask body 10 is provided from a dimensionally stable plastic and the mask bead 12 is made of an elastomeric plastic, the mask body 10 can include a mask fitting at the transition 132, which is configured and designed, for example, such that the mask bead 12 can be plugged in a respiratory gas-tight manner onto the mask body 10 (not shown). The form of the mask bead 12 shown is to be understood as an exemplary embodiment of a mask bead 12 for the breathing mask 1 according to the invention. Variations of the sealing lip 130 or the contact area 128 of the mask bead 12 are possible. The mask bead 12 is configured and designed by the preferably elastomeric or soft material in such a way that upon use of the breathing mask 1, a change of the area of the receptacle opening 32 defined by the contact area 128 can result. The contact area 128 can also vary during use depending on the facial contour of the user.

The material thickness of the mask bead 12 can be designed to be constant or can vary at least in some areas. Overall, the material thickness of the mask bead 12 is dependent on the point of the face of the user on which the respective area rests. The material thickness is thus to be selected as lower in the area of more sensitive points of the face. In areas which rest on less sensitive points of the face, in contrast, a greater material thickness can be selected to stabilize the mask bead 12. For example, the material thickness of the mask bead 3 in the area of the nose bridge area 125 is from about 0.1 mm to about 0.8 mm, preferably from about 0.3 mm to about 0.5 mm.

The material thickness in the base area 127 of the mask bead 12 can initially be greater coming from the transition 132 and can decrease in the direction of the edge 131 of the mask bead 3. In the area of the transition 132, the material thickness is, for example, from about 0.5 mm to about 3.0 mm, preferably from about 0.7 mm to about 2.5 mm. Toward the edge 131, the material thickness is reduced to from about 0.1 mm to about 0.8 mm. Similar profiles of the material thicknesses can also be formed, for example, in the lateral areas 126 of the mask bead 12.

The mask bead 12 can be configured to press against the chin at least in some areas during use of the breathing mask 1. In particular the base area 127 of the mask bead 12 can in some embodiments press against the face of a user in such a way that the chin is enclosed by the breathing mask 1. Such a chin enclosure can be configured by a special shaping of the base area 127. For example, the base area 127 can be shaped concavely in such a way that during use of the breathing mask 1, the chin of a user is received in the base area 127.

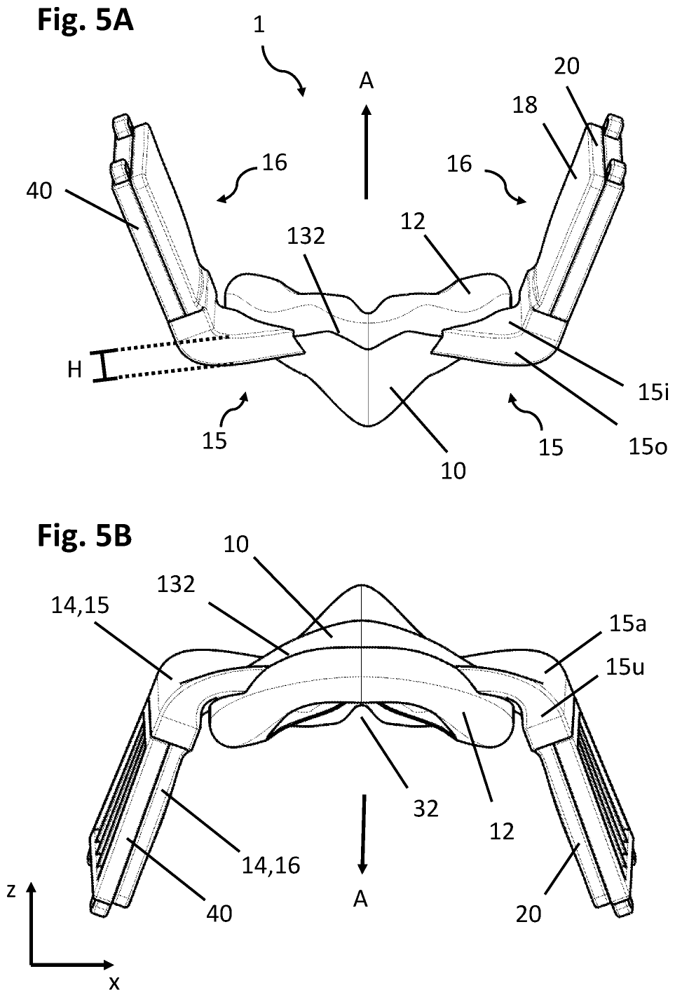

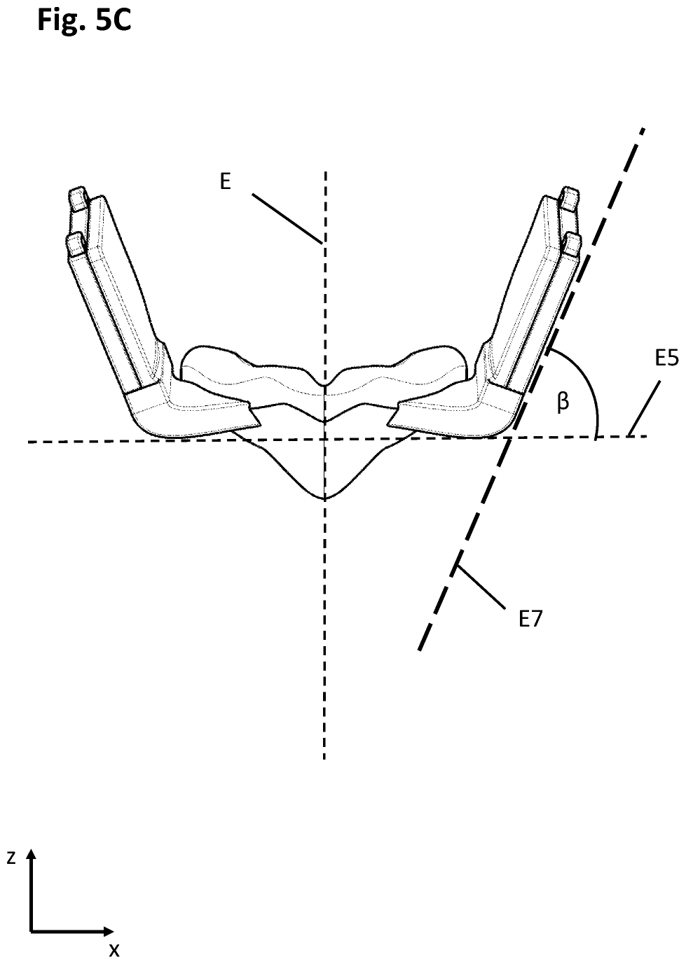

FIG. 5A shows an illustration of a breathing mask 1 with filter elements 40 from above and FIG. 5B shows a breathing mask 1 with filter elements 40 from below. The breathing mask 1 generally comprises two mask wings 14 each having a receiving device 16 and at least one wing duct 15.

In some embodiments, it is also conceivable that the breathing mask 1 includes more than two mask wings 14, for example four or more (not shown). The mask wings 14 are preferably an integral component of the breathing mask 1. Mask wings 14 and mask body 10 are preferably integrally formed. The mask wings 14 are molded onto the mask body 10. The mask wings 14 are preferably arranged on the mask body 10 at the maximum possible distance from the plane of symmetry E (see FIG. 1).

It is apparent from FIGS. 5A/5B that the mask wings 14 comprise two partial areas, namely at least one wing duct 15 and at least one receptacle device 16.

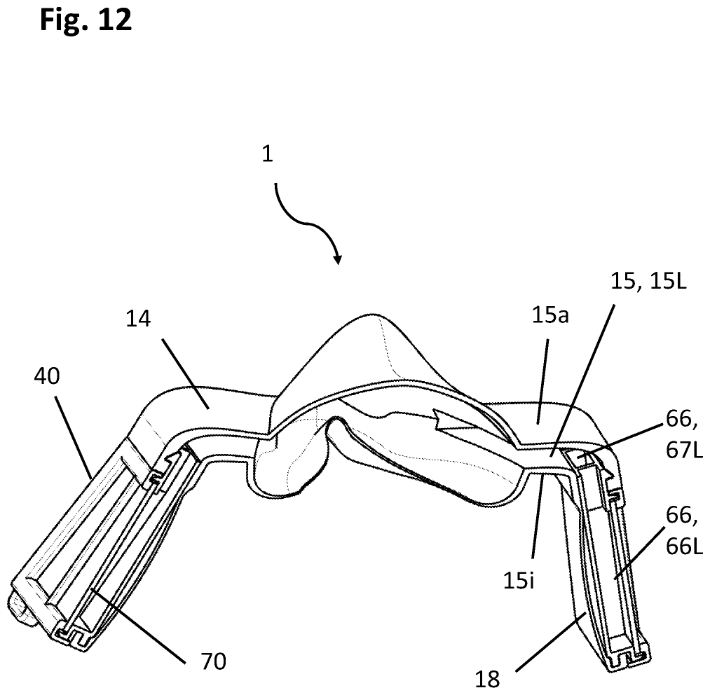

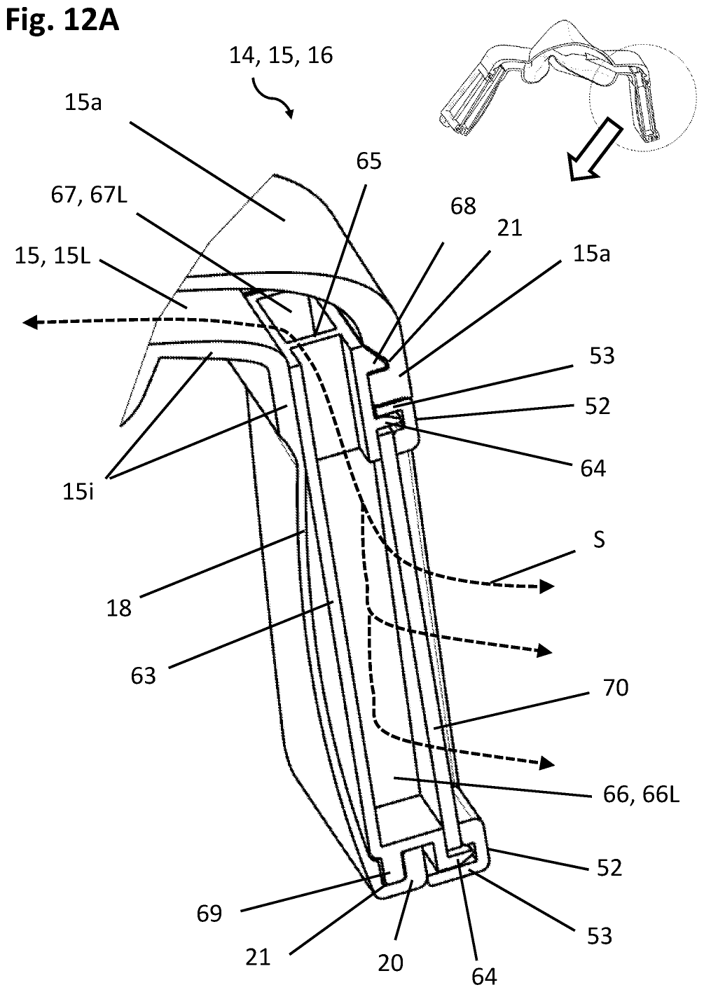

The wing duct 15 can be designed in the form of a hollow body and can include a lumen 15L (see FIG. 12A). The wing duct 15 can have a polygonal, oval, or round cross-section. In one exemplary embodiment, the wing duct 15 can have a square cross-section. The corners can be formed angled or rounded. The mask wing 14 in the area of the wing duct 15 can thus be designed in the form of a square tube.

It is apparent from the exemplary embodiment according to the drawings that the wing duct can include four side walls due to the preferably square cross-section: an outer wall 15a, an inner wall 15i, an upper wall 15o, and a lower wall 15u.

The inner wall 15i (see FIG. 5A) generally faces in the direction toward the user during use of the breathing mask 1. During use, the inner wall 15i faces in the direction of the facial skin of the user. The outer wall 15a (see FIG. 5B) faces away from the user during use of the breathing mask 1.

Outer wall 15a and inner wall 15i are spaced apart from one another. The distance between the inner wall 15i and the outer wall 15a defines a height H of the wing duct 15 (see FIG. 5A).

The outer wall 15a can extend at least partially in parallel to the inner wall 15i. In this case, the height H can be uniform. The height H can also increase and/or decrease. In the specific exemplary embodiment according to the drawings, the height H can extend consistently in large parts and can increase in the direction of the receptacle device 16, for example.

Upper wall 15o (see FIG. 5A) and lower wall 15u (see FIG. 5B) are spaced apart from one another. The distance between the upper wall 15o and the lower wall 15u defines a width B of the wing duct 15 (see FIG. 1B). The width B can be uniform or can increase and/or decrease at least in some sections.

The wing duct 14 can have a first width B1 in the area adjacent to the mask body 10 and can have a second width B2 in the area adjacent to the receptacle device 16. In exemplary embodiments according to the drawings, the first width B1 can be greater than the second width B2 (see FIG. 1B). The width B can thus decrease from the mask body 10 in the direction toward the receptacle device 16. In alternative embodiments, it is also conceivable that the width B remains uniform and/or increases. A decrease of the width B in the flow profile can offer the advantage that the eye area remains free and a free field of view results for the user.

The lumen of the wing duct 15L defines a hydraulic diameter of the wing duct 15. The lumen of the wing duct 15L can have a hydraulic diameter of at least 10 mm per wing duct 15. In one preferred embodiment, the lumen of the wing duct 15L has a hydraulic diameter of at least about 12 mm. In a specific exemplary embodiment, the lumen of the wing duct 15L can have a hydraulic diameter of about 15 mm or more. The hydraulic diameter is designed such that the breathing resistance is as low as possible.

The wing duct 15 can be configured and designed in such a way that the wing duct also does not deform under tensile and/or compressive stress such that the hydraulic diameter changes. For this purpose, the wall thickness of the wing duct 15 can be formed thicker than, for example, the wall thickness of the mask body 10.

In some embodiments, it is also conceivable that the wing duct 15 is made reinforced in areas which have to withstand a particular tensile and/or compressive stress (not shown). The wall thickness of the wing duct 15 can thus be uniform or can be made varying.

In some embodiments, reinforcing elements can reinforce the wing duct 15 in such a way that the lumen of the wing duct 15L cannot deform or cannot deform significantly (not shown). These areas can have a greater wall thickness, for example. These areas can in some embodiments also include additional elements such as ribs or the like. These ribs can be configured and designed to support the wing duct and/or to maintain the distances between the four side walls 15a, 15i, 15o, 15u.

Such supporting and/or spacing ribs can be manufactured, for example, from the same material as the mask wing 14. In some embodiments, such supporting and/or spacing ribs can be manufactured from and/or can include a different material. Such reinforcing elements can be manufactured, for example, from a plastic, in particular from a dimensionally stable plastic. Reinforcing elements made of metal are also conceivable. For example, wires can be incorporated into the base material of the wing duct 15 for reinforcement.

It is apparent from FIG. 5C that the mask wings 14 can be formed angled and/or curved in the area of the wing duct 15. To illustrate this, exemplary planes E5 and E7 are indicated in FIG. 5C. The plane E5 is perpendicular to the plane of symmetry E. The plane E7 is defined by the arrangement of the receptacle device 16 or the filter element 40.

The mask wings 15 can be configured in such a way that the plane E7 is inclined at an angle β of 30° to 90° to the plane E5 when the breathing mask 1 is not subjected to external force. The plane E7 can preferably be inclined at an angle β of at least 45° to the plane E5 when the breathing mask 1 is not subjected to external force. In a specific exemplary embodiment, the plane E7 can be inclined at an angle β of 70° to the plane E5 when the breathing mask 1 is not subjected to external force.

The mask wings 14 comprise, in addition to the wing duct 15, the at least one receptacle device 16. The receptacle device 16 is preferably an integral component of the mask wings 14. Receptacle device 16 and mask wings 14 are preferably integrally formed. An integral embodiment has the advantage of simple manufacturing. The receptacle device 16 is molded onto the wing duct 15 in this case. The wing duct 15 is thus arranged between the mask body and the receptacle device 16. The receptacle device 16 can preferably be arranged at the maximum possible distance from the plane of symmetry E of the breathing mask 1.

In alternative embodiments, it is also conceivable that the receptacle device 16 is a detachable component of the breathing mask 1 and can be detachably connected to the wing duct 15, for example (not shown). Such an embodiment could enable more efficient cleaning, since areas difficult to access can also be reached. Moreover, a multipart design of the breathing mask 1 could facilitate connecting the filter element 40 to the breathing mask 1. For example, it is conceivable that first receptacle device 16 and filter element 40 are connected to one another and are only then attached jointly to the breathing mask 1 or to the wing duct 15. This can facilitate the handling of the breathing mask 1.

The receptacle device 16 of the mask wing 14 is configured and designed to receive the filter element 40. Filter element 40 and receptacle device 16 can be connected, for example, by pressing the dimensionally stable filter element 40 into the elastically deformable receptacle device 16. Receptacle device 16 and filter element 40 are designed at least partially complementary in shape for this purpose.

The two-dimensional basic shape of receptacle device 16 and filter element 40 can be round or polygonal. In one exemplary embodiment, the basic shape of receptacle device 16 and filter element 40 can be square. For example, the basic shapes of receptacle device 16 and filter element 40 are rhombic (not shown) or trapezoidal (see, by way of example, FIG. 2A). A trapezoidal basic shape offers the advantage that the filter element 40 can be inserted on both sides and no distinction into “left” filter element 40 and “right” filter element 40 has to be performed.

It is furthermore apparent from FIG. 5A that the receptacle device 16 comprises at least one receptacle side wall 20. The receptacle side wall 20 is preferably elastically deformable. The receptacle side wall 20 extends around the trapezoidal receptacle device 16, for example. In one exemplary embodiment, the receptacle side wall 20 is embodied peripherally on three sides, wherein no receptacle side wall 20 is formed on the side which faces toward the wing duct 15.

The receptacle device 16 can comprise a receptacle rear wall 18 in addition to the receptacle side wall 20. Upon contact of the breathing mask 1 on the head of the user, the receptacle rear wall 18 can be at least partially facing toward the face and/or pressing against the face.

The receptacle rear wall 18 can be adjacent to the inner wall 15i of the wing duct 15. The receptacle rear wall 18 can be connected to the inner wall 15i of the wing duct 15. In the specific exemplary embodiment according to the drawings, inner wall 15i and receptacle rear wall 18 are integrally formed and face in the direction of the user. The receptacle rear wall 18 can stabilize the receptacle device 16 as such, which facilitates receiving the filter element 40 in the receptacle device 16.

In some embodiments, the receptacle device 16 can also be designed without receptacle rear wall 18. The receptacle device 16 then consists solely of the receptacle side wall 20. Upon contact of the breathing mask 1 on the head of the user, the filter device 40 can then be facing at least partially toward the face and/or pressing against the face. An embodiment without receptacle rear wall 18 offers the advantage of a material saving, which in turn reduces the weight of the breathing mask and can reduce production costs.

The receptacle device 16 is configured in such a way that it can accommodate the filter element 40. When the filter element 40 is connected to the shape-complementary receptacle device 16, at least a section of the filter element 40 is located within at least one area of the lumen of the wing duct 15L. In one exemplary embodiment, a side of the square filter element is arranged at least in some sections in the lumen of the wing duct 15L. The other three sides of the filter element 40 are arranged inside the receptacle side wall 20. The filter element can thus be introduced into the elastically deformable receptacle device 16 in such a way that the filter element 40 is pressed in within the receptacle side walls 20 that are peripheral on three sides.

For this purpose, the receptacle device 16 can be embodied so that it is at most, for example, as large as the filter element 40. For example, the receptacle device 16 is made slightly smaller than the filter element 40. The filter element 40 can then be pressed into the elastically formed receptacle device 16 to establish a usage state. The receptacle side wall 20 initially slightly deforms in this case and can return to its original shape after receiving the filter element 40. The filter element 40 is thus held in the receptacle device 40 under slight tension.

The side of the receptacle side wall 20 opposite to the wing duct 15 can moreover include a locking recess 21 in order to lock the filter element 40 in the receptacle device 16 (see FIG. 12A).

Pressing the filter element 40 into the receptacle device 16 is preferably reversible. The filter element 40 can thus generally be removed from the receptacle device 16 again, for example for cleaning the breathing mask 1 or for changing the filter material 70. In some embodiments, it is also conceivable that the filter material 70 can also be changed when the filter element 40 is connected to the breathing mask 1.

When the filter element 40 is connected to the breathing mask 1, mask body 10 and mask bead 12 and mask wing 14 with the wing ducts 15 and filter element 40 define a respiratory gas space 2, which is essentially respiratory gas-tight during use of the breathing mask 1. A respiratory gas flow away from the mouth and nose of the user and toward the mouth and nose of the user is thus exclusively possible during use of the breathing mask 1 through the filter element 40 and in particular through the filter element 70.

The respiratory gas space 2 has a volume which is sufficiently small that CO₂ is washed out from the respiratory air of the user. The respiratory gas space 2 has, for example, a volume of at most 500 ml. The volume of the respiratory gas space 2 has, for example, a volume of 50 ml to 500 ml. The volume of the respiratory gas space 2 during use of the breathing mask 1 is preferably at most 120 ml.

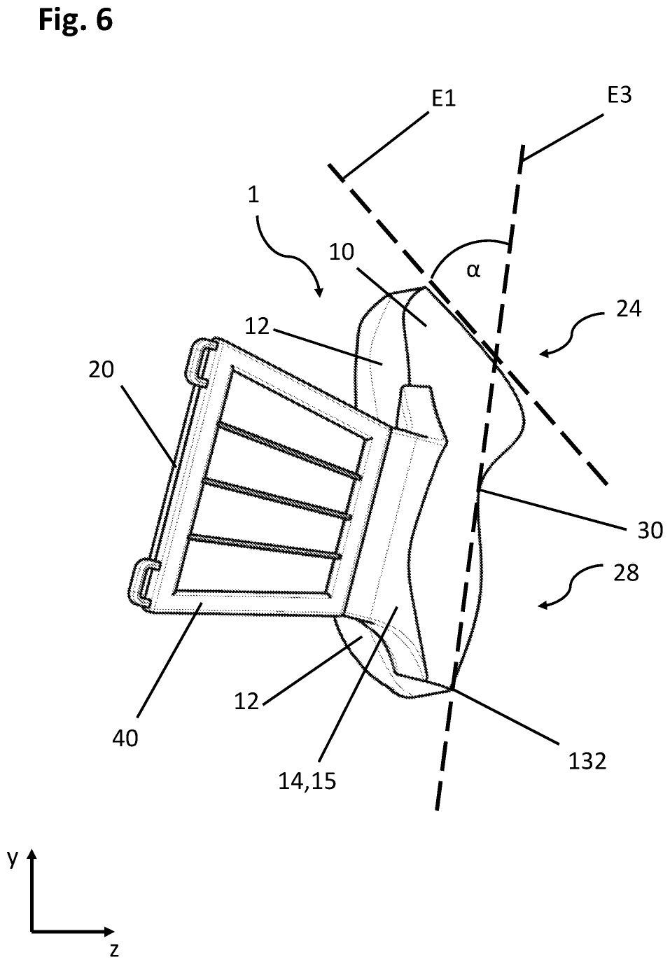

FIG. 6 shows an illustration of a breathing mask 1 with filter elements 40 from the side. The mask body 10 is generally manufactured from a polymer. The mask body 10 can be manufactured from a dimensionally stable and/or elastomeric polymer. In one preferred exemplary embodiment, the mask body 10 is manufactured from silicone. Silicone is an elastomeric polymer which is dimensionally stable as such, but can be elastically deformable under tensile and/or compressive load. In addition to silicone as the base material of the mask body 10, further materials can be used. For example, it is conceivable that materials for reinforcing and/or stiffening and/or cushioning are introduced and/or applied into or to the silicone.

It is apparent from FIG. 6 that the mask body 10 is preformed. The mask body 10 can essentially include a nose area 24 and a mouth area 28. The nose area 24 essentially covers the nose bridge and the nose of the user in a usage state on the head 90 of a user. The mouth area 28 essentially spans at least the mouth of the user in a usage state on the head 90 of a user.

Between nose area 24 and mouth area 28, there is an attachment area 30, which can be designed and configured as a predetermined buckling point. The attachment area 30 can for this purpose have a material thickness and/or material quality varying from the nose area 24 and/or from the mouth area 28. For example, nose area 24 and mouth area 28 can have a higher material thickness than the attachment area 30.

In some embodiments, nose area 24 and mouth area 28 can alternatively or additionally have an essentially identical material property and the attachment area 30 can have different material properties in relation thereto. For example, the Shore hardness and thus the maximum extensibility can be formed differently in the different areas 24, 28, 30.

In some embodiments, reinforcing elements (not shown) can be arranged in the mouth area 28 and/or in the nose area 24. Such reinforcing elements can be configured and designed to absorb forces which act through the head attachment via the head fastening straps on the breathing mask 1. The reinforcing elements can be formed in the form of material thickenings. The reinforcing elements can also be additionally introduced elements. For example, reinforcing material can be made of a hard polymer and/or a metal and/or a thermoplastic and/or a fabric. The reinforcing elements can be designed in the form of wires, bows, fibers, rods, strips, or the like. In advantageous embodiments, the reinforcing elements can be designed as deformable elements, so that the breathing mask 1 can be adapted individually to the head shape of the respective user.

FIG. 6 shows how nose area 24 and mouth area 28 of the mask body 10 are arranged in relation to one another. Exemplary planes E1 and E3 are indicated for this purpose. The nose area 24 defines a plane E1 in this case. The mouth area 28 defines a plane E3, wherein this intersects the attachment area 30 located in the plane of symmetry E and the transition 132 located in the plane of symmetry E at the base of the breathing mask.

The mask body 10 is configured in such a way that the plane E1 is inclined at an angle α from about 30° to about 70° to the plane E3 when the breathing mask 1 is not subjected to external force. The plane E1 can preferably be inclined at an angle α of at least about 40° to the plane E3 when the breathing mask 1 is not subjected to external force. In a specific exemplary embodiment, the plane E1 can be inclined at an angle α of about 48° to the plane E3 when the breathing mask 1 is not subjected to external force.

Because the attachment area 30 can be designed and configured in such a way as a predetermined buckling point, the advantage of better adaptability of the breathing mask 1 to the face of a user results. In users having a flat nose bridge, the breathing mask 1 can remain in its original preformed state. In users having a pronounced or high nose bridge, the breathing mask can adapt itself to the contour of the face.

Upon application of the breathing mask 1 to the head, a high or pronounced nose bridge of the user can displace the nose area 24 in such a way that the angle α between plane E1 and plane E3 can change. The angle α between plane E1 and plane E3 can change under force action.

The mask body 10 is configured in such a way that the angle α can be reduced by at least about 5° upon application of the breathing mask 1 to the head of a user. The angle α can be reduced by up to about 25° upon application of the breathing mask 1 to the head of a user.

The nose area 24 can in some embodiments include reinforcing and/or cushioning areas. The nose area 24 can optionally include stiffening elements, which are also deformable and can be arranged, for example, transversely to the axis of symmetry E. For example, deformable bows made of wire or a comparable material can be introduced so that by deforming the bows, an optimum adaptation of the mask body 10 to the individual nose bridge can take place.

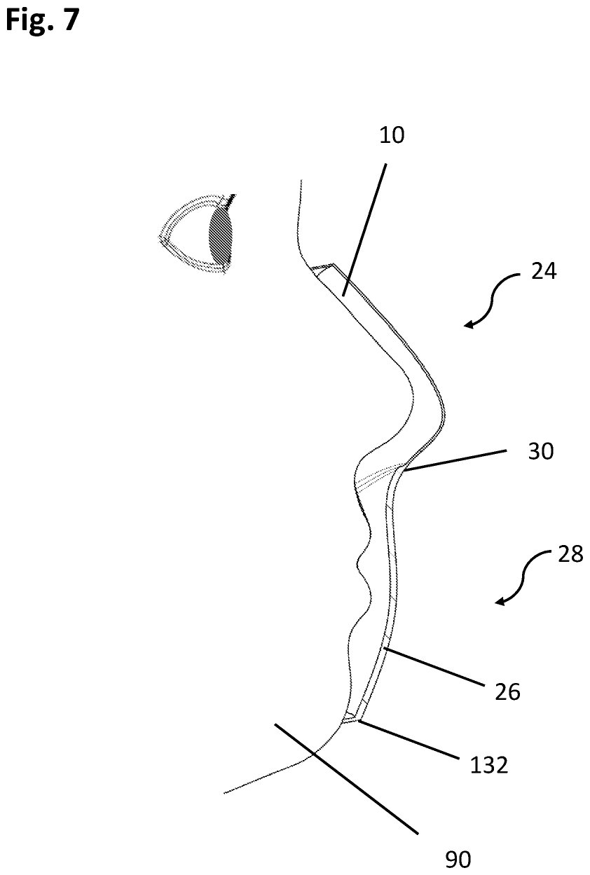

FIG. 7 shows a simplified view of a breathing mask 1 in cross-section upon contact on the head 90 of a user. The wall thickness of the mask body 10 can be made uniform or varying. In the exemplary embodiment shown in FIG. 7, it is clear from the cross-section that the wall thickness of the mask body 10 can be formed having different thicknesses in different areas 24, 28.

Thus, for example, the nose area 24 can have a lesser wall thickness in relation to the remaining mask body 10. The area of the nose is generally a sensitive area in the phase, so that a lesser wall thickness can have a positive effect here on the wearing comfort. In contrast, the mouth area 28 can, for example, have an increased wall thickness in relation to the nose area 24. The breathing mask 1 as such can be stabilized by the increased wall thickness in the mouth area 28.

The wall thickness in the nose area 24 can be in a range from about 0.1 mm to about 3 mm. In preferred exemplary embodiments, the wall thickness in the nose area 24 can be in a range from about 0.3 mm to about 1.5 mm. In one specific exemplary embodiment, the wall thickness in the nose area 24 is about 0.7 mm.

The wall thickness in the mouth area 28 can be in a range from about 0.1 mm to about 5 mm. In preferred exemplary embodiments, the wall thickness in the mouth area 28 can be in a range from about 1 mm to about 3 mm. In one specific exemplary embodiment, the wall thickness in the mouth area 28 is about 1.5 mm.

Nose area 24 and mouth area 28 each have an essentially constant wall thickness in the specific exemplary embodiment according to the drawings. Varying wall thicknesses within the nose area 24 and/or within the mouth area 28 are also conceivable in some exemplary embodiments.

The attachment area 30 is arranged between the nose area 24 having a preferably low wall thickness and the mouth area 28 having a preferably increased wall thickness in contrast to the nose area 24. The wall thickness in the attachment area 30 can be in a range from about 0.1 mm to about 5 mm. In the attachment area 30, the nose area 24 and the mouth area 28 can meet one another directly, so that the wall thickness jumps between the wall thickness of the nose area 24 and the wall thickness of the mouth area 26.

Because the mask bead 12 presses with its contact area 128 embodied as a sealing lip 130 in a respiratory gas-tight manner against the facial skin, mask body 10 and face of the user are spatially separated from one another. The smaller the distance between mask body 10 and the face of the user, the smaller is the dead space volume. The dead space volume can be made very small by the shaping of the breathing mask 1. The dead space volume is the volume which forms between the breathing mask 1 and the surface of the face in a usage state. It is apparent from FIG. 7 that the breathing mask 1 forms an equidistant surface to the face of the user in large parts, in particular in each case in the nose area 24 and in the mouth area 28. The breathing mask 1 according to the invention thus offers the advantage that due to the small dead space volume, the risk of CO₂ rebreathing is very low for the user. The dead space volume of the breathing mask 1 is less than about 200 ml in preferred exemplary embodiments. For example, the dead space volume of the breathing mask 1 is less than about 120 ml.

The distance between the mask body 10 and the face of the user is on average less than about 20 mm. In some embodiments, the distance between the mask body 10 and the face of the user is on average less than about 15 mm. For example, the distance between the mask body and the face of the user is on average less than about 10 mm.

The design according to the invention of the mask body 10 can also be transferred to other breathing masks. For example, it is conceivable that breathing masks for ventilation and/or for respiratory assistance are equipped with the mask body 10 according to the invention to obtain an optimum adaptation to the facial contour of the user.

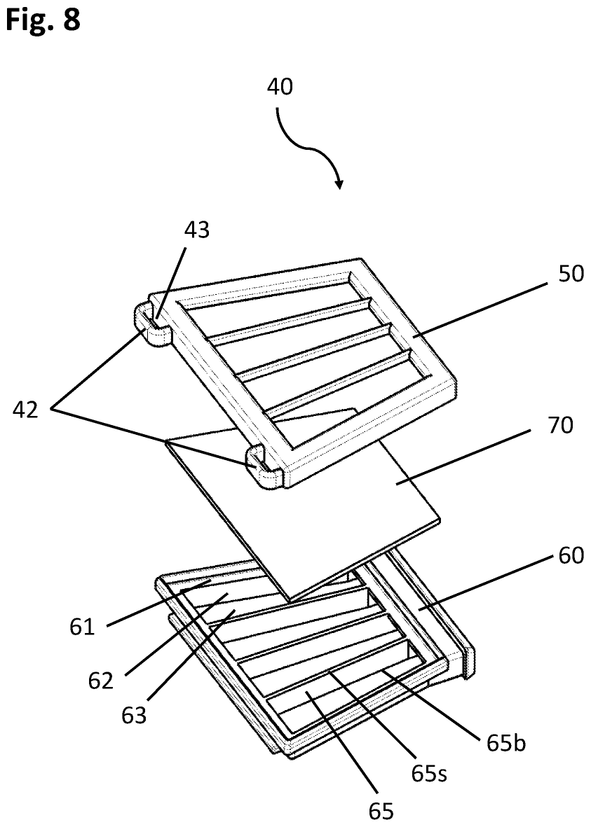

FIG. 8 shows an exploded view of a filter element 40. It is apparent from FIG. 8 that the filter element can comprise three main components, namely a front part 50, a rear part 60, and a filter material 70. The basic shape of the filter element 40—and thus also of its components 50, 60, 70—can preferably be made essentially square, for example trapezoidal. A rhombic, square, round, semicircular, or free-form design is also conceivable (not shown). The filter element 40 is designed complementary in shape to the receptacle device 16, so that the filter element 40 can be pressed into the receptacle device 16 to establish a usage state of the breathing mask 1.

The particle-filtering filter material 70 can be formed flat in a simple embodiment. The filter material 70 can be used in one layer or multiple layers. The filter material 70 can also be made corrugated or folded like an accordion in some embodiments. The filter surface can thus be increased without enlarging the area of the filter element 40. In a further embodiment, the filter material 70 can consist, for example, of a gas-permeable container or bag, which is filled with a filter material, for example activated carbon.

The filter material 70 can have a height, a width, and a thickness. The filter material 70 can be made essentially flat. The thickness of the filter material 70 can be made multiple times less than its height and width. The height and width of the filter material 70 form an area of the filter material 70, through which the gas exchange can take place at least in some areas during use of the breathing mask 1.

The filter material 70 can have an area of at least about 5 cm². In some embodiments, the area of the filter material 70 can be at least about 30 cm². In one preferred embodiment, the area of the filter material 70 can be at least about 50 cm².

The filter material 70 can be arranged between the front part 50 and the rear part 60. The filter element 40 is configured in such a way that the filter material 70 is to be removed easily and quickly. The filter material 70 can then be replaced and/or cleaned.

Front part 50 and rear part 60 can be formed in two parts and complementary in shape for this purpose. Front part 50 and rear part 60 can be designed in such a way that they can be reversibly connected to one another. The connection of front part 50 and rear part 60 can take place, for example, via pressing in. For example, front part 50 and rear part 60 can be designed in such a way that the front part 50 can be pressed reversibly at least in some areas into the rear part 60 and vice versa. In some embodiments, the front part 50 can also be connected to the rear part 60 without pressing in and can be fastened, for example, via additional holding elements, such as small hooks, on the rear part 60.

In an alternative embodiment (not shown), it is also possible that front part 50 and rear part 60 are permanently connected to one another at least in some areas. It is conceivable, for example, that front part 50 and rear part 60 are connected to one another via a folding connection, for example a hinge, on one side. The filter element 40 as a whole with its front part 50 and its rear part 70 can thus be designed so it can be folded open, in order to insert or replace the filter material 70.

The filter element 40 is in a usage state when front part 50 and rear part 70 are connected to one another and enclose the filter material 70.

In one specific preferred exemplary embodiment, the breathing mask 1 consists of a silicone part, in which preferably two filter elements 40 made of an essentially dimensionally stable plastic are inserted, which each contain a filter material 70. The head attachment of the breathing mask 1 can take place via simple straps. For this purpose, the breathing mask 1 can comprise at least one strap bracket 42.

The strap bracket 42 can be arranged on the filter element 40 (see FIG. 8 et seq.) or on the mask wings 14 (not shown). An arrangement of the strap bracket 42 on the dimensionally stable filter element 40 offers the advantage that the strap is held stably and the breathing mask 1 does not experience significant shape change even under tensile and/or compressive force due to the strap.

The strap bracket 42 can be formed, for example, in the form of a projection, which is provided with at least one feedthrough opening 43. A head fastening strap (not shown) can be fed through the feedthrough opening 43 and, for example, knotted or clamped with the strap mount 42. Clamping can be achieved, for example, in that the feedthrough opening 43 is embodied conically or funnel-shaped and is arranged tapering in the direction away from the user, but not closing. Simple threading can be enabled by the funnel shape, but the opposite pulling direction can be blocked or at least made much more difficult. Alternatively, the head fastening strap can also be equipped with a hook-and-loop closure, so that the head fastening strap is fastened to itself as such and a length adjustment is possible.

A firm seat of the breathing mask 1 on the face of the user can be ensured using the head fastening strap. In one exemplary embodiment, the head fastening strap is an elastic strap, which extends over or behind the head of the user in a stretched state and then presses the breathing mask 1 slightly against the face of the user in a less stretched state, in order to terminate it respiratory gas-tight in relation to the face.

In one preferred embodiment, the filter element 40 can comprise the at least one strap bracket 42. In the exemplary embodiment according to the drawings, the strap bracket is arranged on the front part 50 of the filter element 40 (see FIG. 8 et seq.). An arrangement on the rear part 60 is also conceivable in alternative embodiments.

The breathing mask 1 contains at least two, preferably at least four or more strap brackets 42. In one specific exemplary embodiment, two strap brackets 42 are arranged in each case in each of the two mirror-symmetrical halves of the breathing mask 1. Two strap brackets 42 are preferably arranged in each case on one filter element 40 in each case. In one specific exemplary embodiment, the two strap brackets 42 are arranged spaced apart from one another on the front part 50 of the filter element 40.

To prevent the breathing mask 1 from deforming undesirably under the compressive and/or tensile forces, it is also conceivable in some embodiments that reinforcing elements are introduced into the breathing mask 1, which prevent a deformation under force action. For example, reinforcing bows can be arranged in an area which presses below the nose in a usage state. These can thus be arranged between the two upper strap brackets 42 and can absorb the forces due to the transverse connection. Alternatively or additionally, reinforcing bows can also be arranged in an area which presses against or below the chin in a usage state, since the force conduction can extend through the lower mask edge. A chin enclosure at the base of the breathing mask 1 is also conceivable (not shown).

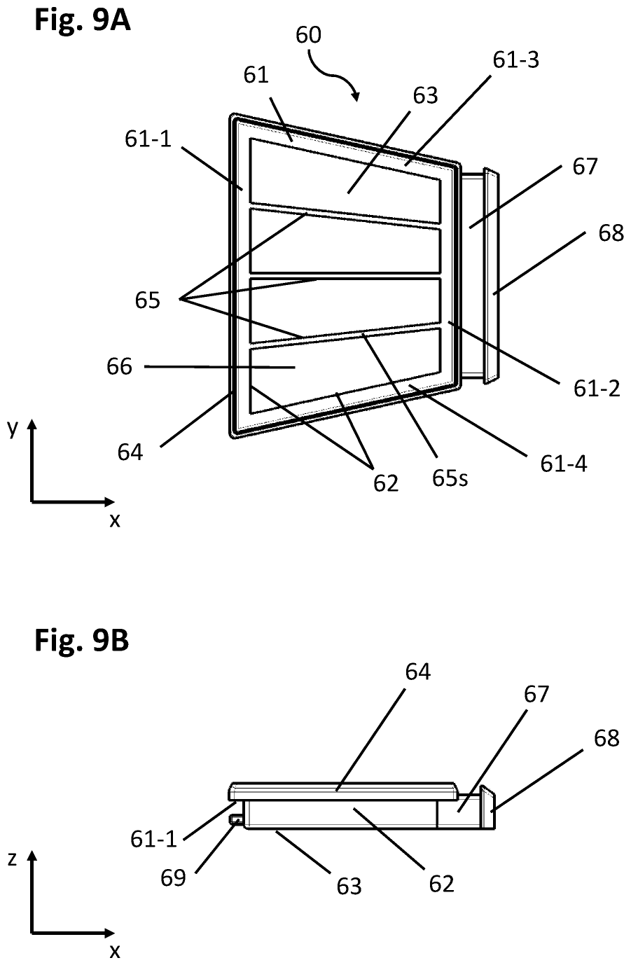

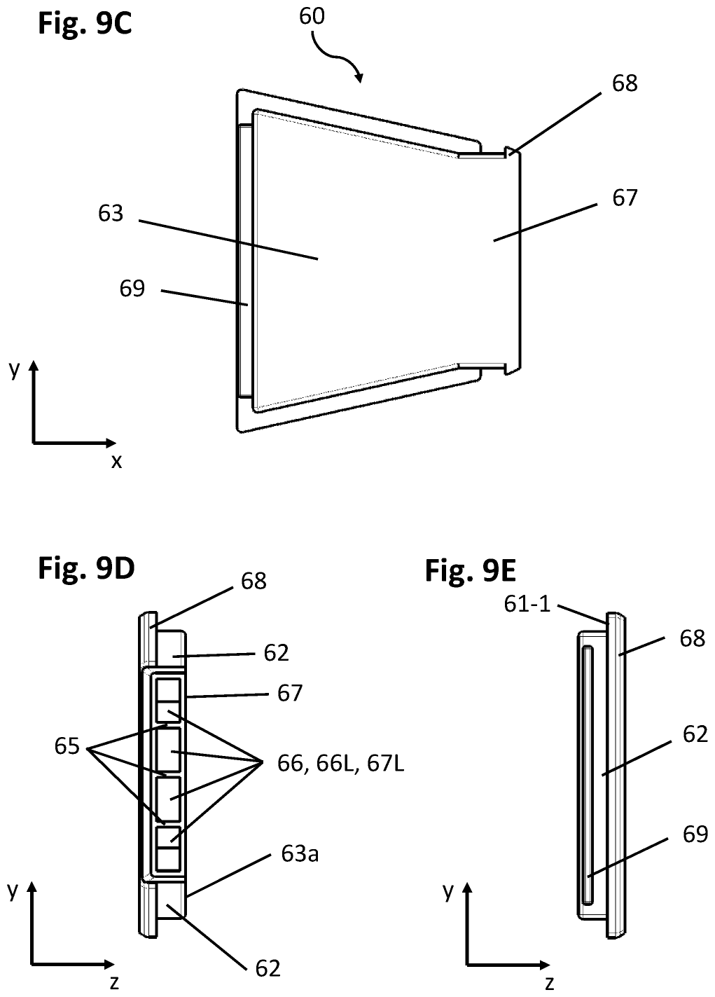

FIGS. 9A-E show a rear part 60 of a filter element 40 from different perspectives. The coordinate systems shown are used to illustrate the alignment of the views.

The rear part 60 comprises at least one air space, which can be formed in the form of at least one duct 66. The duct 66 is delimited by a rear wall 63 of the rear part 60 and—at least in some areas—by at least one side wall 62. The side wall 62 is generally arranged perpendicular to the rear wall 63. Rear wall 63 and side wall 62 represent a base body of the rear part 60 and define the duct 66 at least in some areas. Rear wall 63 and side wall 62 can have a smooth surface in simple embodiments. In some embodiments, structured surfaces are conceivable in order to positively influence the flow properties of the duct 66 (not shown).

The rear part 60 can moreover include at least one duct wall 65. The duct walls 65 are generally arranged perpendicular to the rear wall 63. The duct 66 can be divided into multiple subunits by the duct walls 65.

The rear part 60 can contain arbitrarily many duct walls 65, for example one or more, preferably at least two. In one specific embodiment according to the drawings, the rear part 60 can include, for example, three duct walls 65, which divide the duct 66 into four subunits. Embodiments having more than three duct walls 65 are also possible.

The duct walls 65 have a base 65b and an opposite end face 65s (see FIG. 8). The duct walls 65 generally extend linearly from their base 65b to their end face 65s. The duct walls 65 can be arranged with their base 65s on the rear wall 63 of the rear part. Duct walls 65 and rear wall 63 can then be formed fixedly connected to one another. It is not absolutely necessary to connect the duct walls 65 to the rear wall, however, the duct walls provide additional stability. The ducts 63 are formed open toward the end face 65s and can be covered at least in some areas by placement of the filter material 70.

The rear part 60 can comprise a support surface 61 (see FIG. 9A). The support surface 61 can extend in parallel to the rear wall 63 and is spaced apart therefrom. The support surface 61 can include an outer contour corresponding to the filter material 70, namely preferably a polygonal outer contour, particularly preferably a square. For example, the outer contour of the support surface 61 is formed trapezoidal. The filter material 70 can be laid on the support surface 61 and on the end faces of the duct walls 65s of the rear part 60 to establish a usage state and can then be connected to the front part 50. The duct walls 65 can therefore be configured and designed with their end faces 65s in particular as an additional support option for the filter material 70.

The duct walls 65 can also have other shapes in some embodiments. For example, the duct walls 65 can be designed in the form of columns or steles and accordingly can only provide additional support options for the filter material 70 at points. Dividing the duct 65 into subunits would then not be provided (not shown).

The support surface 61 can contain additional elements in some embodiments. For example, the support surface 61 can include additional elements which span the duct 66 in the form of ribs or grids, to prevent the filter material 70 from being able to be pressed into the rear part 60 or into the duct 66 (not shown).

The rear part 60 can preferably comprise an edge 64. The edge 64 is perpendicular to the support surface 61 and can delimit the support surface 61 to the outside. The filter material 70 can preferably be arranged precisely fitted on the support surface 61, wherein the edge 64 can hold the filter material 70 in position. In one preferred embodiment, the edge 64 is formed as tall as the filter material 70 is thick here.

The rear wall 63 and the support surface 61 or the end faces of the duct walls 65s are arranged and formed spaced apart from one another in such a way that the air space or the ducts 66 are formed after placement of the filter material 70 (see FIG. 8). The side wall 62 can be formed peripherally on three sides around the duct 66.

In one preferred embodiment, the rear part 60 is essentially trapezoidal. In particular, support surface 61 and edge 64 are formed trapezoidal. The trapezoidal support surface 61 can form a generally planar rectangle having four sides 61-1, 61-2, 61-3, and 61-4 in this case. The support surface 61 can include two base sides 61-1, 61-2 lying in parallel to one another. The parallel base sides 61-1, 61-2 can be formed in different lengths. For example, the base side 61-1 can be formed longer than the base side 61-2. The two sides of the support surface 61-3, 61-4 adjoining the parallel base sides 61-1 and 61-2 cannot extend in parallel due to the base sides 61-1 and 61-2 of different lengths.

The duct 66 or the ducts 66 are open toward the side located adjacent to the shorter base side 61-2 and can adjoin a tunnel element 67 there (see FIG. 9). The tunnel element 67 can be formed in one exemplary embodiment as an additional element protruding out of the trapezoid shape. The tunnel element 67 can be adjacent to the base side 61-2 of the support surface. The tunnel element 67 is made tubular. The tunnel element 67 is preferably formed as a square tube, which is open toward both ends. The tunnel element 67 includes a lumen 67L (see FIG. 9D). The lumen of the tunnel element 67L and the lumen of the ducts 66L are connected to one another to conduct (respiratory) gas.

The duct walls 65 can run through the lumen of the tunnel element 67L in some embodiments, so that the lumen of the tunnel element 67L can accordingly be divided into partial lumens.

The lumen of the tunnel element 67L defines a hydraulic diameter which can essentially correspond to that of the wing duct 15.

In embodiments having duct walls 65, the entirety of the partial lumens of the tunnel element 67 defines a hydraulic diameter which can essentially correspond to that of the wing duct 15.

The lumen of the tunnel element 67L can have a hydraulic diameter of at least about 10 mm. In one preferred embodiment, the lumen of the tunnel element 67L has a hydraulic diameter of at least about 12 mm. In one specific exemplary embodiment, the lumen of the tunnel element 67L can have a hydraulic diameter of about 15 mm or more.

At least one locking projection 68 can be formed on the tunnel element 67. It is apparent from FIGS. 9A and 9B that the locking projection 68 can be formed, for example, as a three sided projection. The filter element 40 can be locked, for example, via the locking projection 68 after being received in the receptacle device 16 of the mask wing 14.

Alternatively or additionally to the locking projection 68, the rear part 60 and/or the front part 50 can include a locking rail 69 (see FIGS. 9B, 9C, 9E). The filter element 40 can alternatively or additionally be locked via the locking rail 69 after being received in the receptacle device 16, for example. For this purpose, the receptacle device 16, in particular the receptacle side wall 20, can include corresponding locking recesses 21 (see FIG. 12A). The locking rail 69 can be formed on the side wall 62 of the rear part 60. It is conceivable here in some embodiments that the locking rail 69 extends over the entire length of the side wall 62 (not shown). In the specific exemplary embodiment according to the drawings, the locking rail 69 can only be formed on the side wall 62 which is adjacent to the longer base side of the support surface 61-1 (see FIG. 9E). The locking rail 69 can be formed as an elongated rail according to the drawings. In some embodiments, the locking rail 69 can also have any other suitable shape. For example, it is also conceivable in simpler embodiments that the locking rail 69 is formed as a punctiform locking projection, for example in the form of a button or hook.

FIG. 9D shows a side view of the rear part 60 looking toward an open end of the tunnel element 67. It is apparent from FIG. 9D that the lumen of the channel 66L and/or the lumen of the tunnel element 66L can be divided by the duct walls 65. In this specific exemplary embodiment, the duct 66 and the tunnel element 67 are divided into four partial sections.

The air exchange can take place through the tunnel element 67. In a usage state, the air exchange takes place through the tunnel element 67 and the duct 66 of the rear part 60. The open end of the tunnel element 67 faces toward the wing duct 15 in a usage state. Ducts 66, tunnel element 67, and wing duct 15 are then connected to one another to conduct (respiratory) gas.

In some embodiments, the duct 66 in the filter element 40 can be formed getting flatter in the direction from the tunnel element 67 toward the end (not shown). A material saving can thus be achieved and the breathing mask 1 can be made more delicate overall.

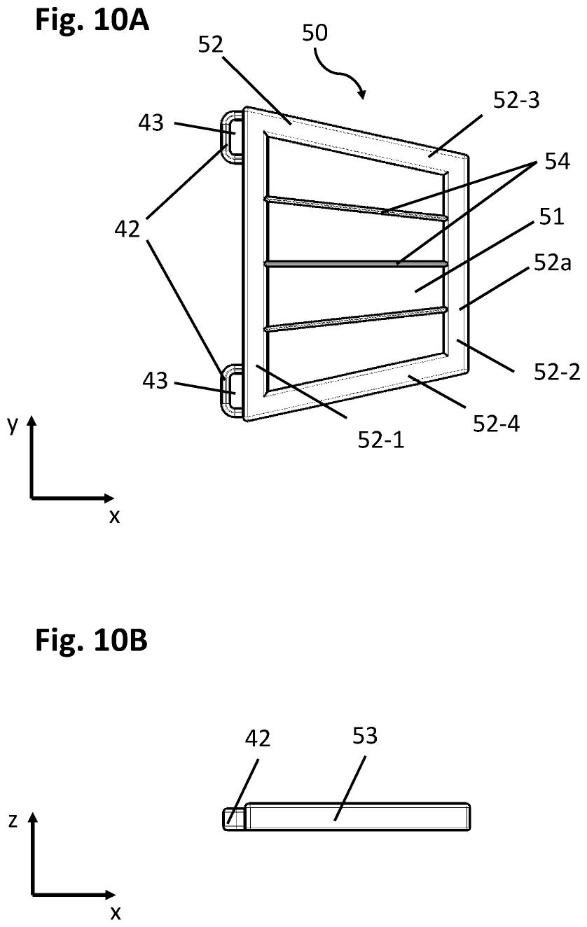

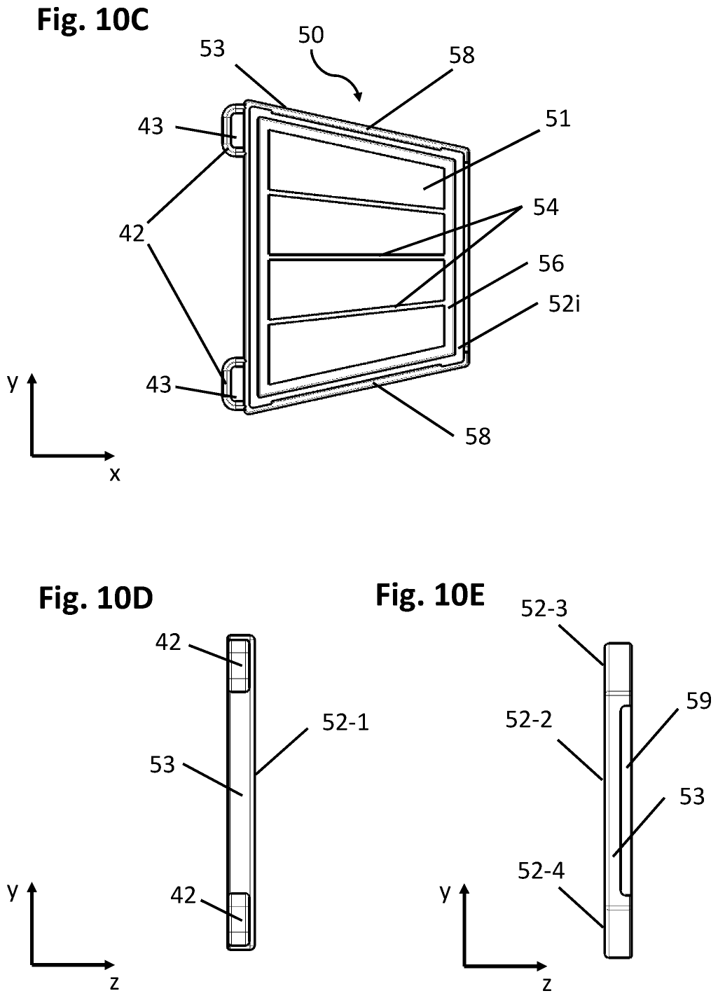

FIGS. 10A-E show a front part 50 of a filter element 40 from different perspectives. The coordinate systems shown are used to illustrate the alignment of the views. The front part 50 comprises a frame 52. The frame 52 delimits the front part 40 to the outside. The frame 52 accordingly has, like the filter element 40 as a whole and like the rear part 60, a polygonal shape, preferably a quadrilateral. The frame 52 is particularly preferably made trapezoidal.

In one preferred embodiment, the frame 52 is trapezoidal and thus forms a generally planar quadrilateral having four sides 52-1, 52-2, 52-3, and 52-4. The frame 52 can include two base sides 52-1, 52-2 lying parallel to one another. The parallel base sides 52-1, 52-2 can be formed having different lengths. For example, the base side 52-1 can be made longer than the base side 52-2. The at least one strap bracket 42 can be molded on adjacent to the base side 52-1. Preferably, two strap brackets 42 are molded on adjacent to the base side 52-1. The two strap brackets 42 are preferably located at the maximum possible distance from one another at the ends of the base side 52-1. The two sides of the frame 52-3, 52-4 adjoining the parallel base sides 52-1 and 52-2 cannot extend in parallel due to the base sides 52-1 and 52-2 of different lengths.

The frame 52 having its four sides 52-1, 52-2, 52-3, 52-4 defines an opening 51 in the inner surface of the front part 50, through which the air exchange can take place. In a usage state, the air exchange takes place through the opening 51 of the front part 50 and through the filter material 70.

The opening 51 can be formed without further elements (not shown). In one preferred embodiment, at least one rib 54 can be arranged continuously within the frame 52 and thus the opening 51. The ribs 54 can be used, inter alia, to hold the filter material 70 between the front part 50 and the rear part 60 and secure it against slipping out. Moreover, an additional stability can be given to the frame 52 by the ribs 54.

The ribs 54 can be arranged arbitrarily and can be embodied in an arbitrary number. In some embodiments, the ribs 54 can also be absent (not shown). In an exemplary embodiment according to the drawings, for example, three ribs 54 are not arranged in parallel to one another, for example. The ribs 54 can also be embodied in other courses. The ribs 54 can in some embodiments also be arranged in parallel and/or in such a way that they meet or cross (not shown) centrally in the opening 51 defined by the frame 52.

The ribs 54 of the front part 50 are preferably formed equivalently to the duct walls 65 of the rear part 60. This means that the duct walls 65 and the ribs 54 are preferably arranged in such a way that they meet one another in a usage state of the filter element 40 with their end faces.

The end faces 65s of the duct walls 65 and the end faces of the ribs 54 are only separated from one another here by the filter material 70. The duct walls 65 and the ribs 54 can clamp the filter material 70 at least in some areas. The filter material 70 can thus be held in position and slipping of the filter material 70 can be substantially prevented.

The frame 52 includes an outside 52a and an inside 55i, wherein the inside 52i faces toward the filter material 70 in a usage state of the filter element 40. In FIG. 10A, the outside 52a is shown. The outside 52a can be made smooth and/or structured. In a simple exemplary embodiment, the surface of the outside 52a is made substantially smooth. In some embodiments, it is conceivable that the outside 52a has a structured surface, in order to give the user tactile feedback about the direction in which the front part 50 can be inserted into the filter element 40 (not shown).

It is apparent from FIG. 10B that the front part 50 comprises a lateral surface 53. The lateral surface 53 is molded on the frame 52 and gives the front part 50 a three-dimensional structure. The at least one strap bracket 52 can be molded on the lateral surface 53.

It is apparent from FIG. 10D that the strap bracket 42 is provided in a double embodiment in this specific exemplary embodiment. The two strap brackets 42 can preferably be molded at the maximum possible distance from one another at the ends of the lateral surface 53, which are adjacent to the base side 52-1. In some embodiments, one or more strap brackets 42 can also be comprised. In an embodiment having a strap bracket 42, a placement in the center of the lateral surface 53 could be advantageous in order to cause an optimum force action on the breathing mask 1.

The inside 52i is shown in FIG. 10C. The inside 52i can include a support surface 56. The support surface 56 can be formed essentially peripherally around the opening 51. The support surface 56 can be formed raised in relation to the inside 52i. For example, the support surface 56 and the end face of the ribs 54 are formed at one height level and in such a way that the filter material 70 can rest on the support surface 56 and the end faces of the ribs 54. In particular, the ribs 54 prevent the filter material 70 from being able to fall and/or be pressed out of the breathing mask 1 through the frame 52 or through the opening 51 in this case.

At least one lock 58 can be molded on the lateral surface 53. The front part 50 can be connected to the rear part 60 via the lock 58 after a connection by means of pressing in. The front part 50 can then only be separated from the rear part 60 again with application of a certain pressure.

It is apparent from FIG. 10E that the lateral surface 53, which is arranged adjacent to the base side 52-2 can include at least one recess 59. The recess 59 is configured and designed to at least partially enclose areas of the rear part 60.

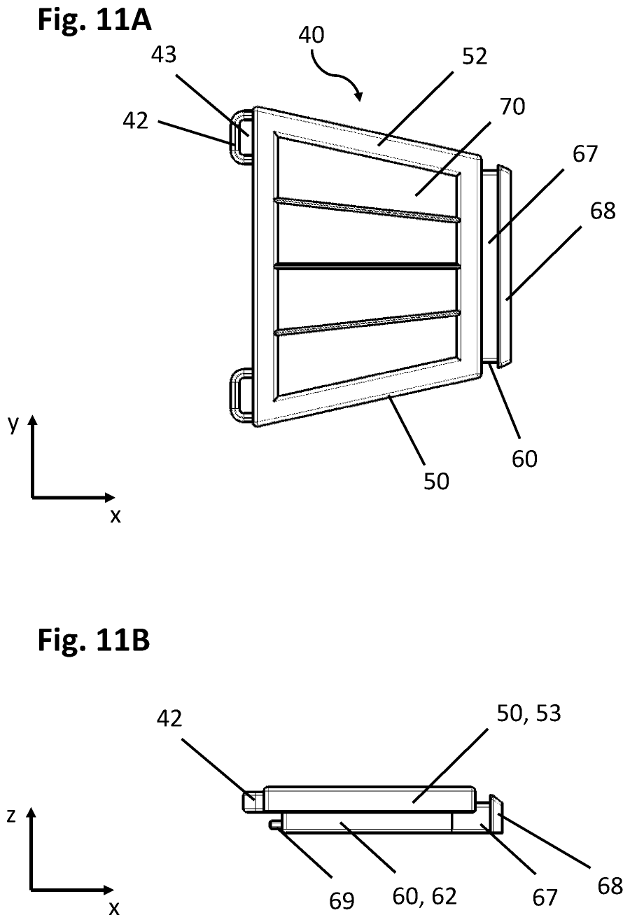

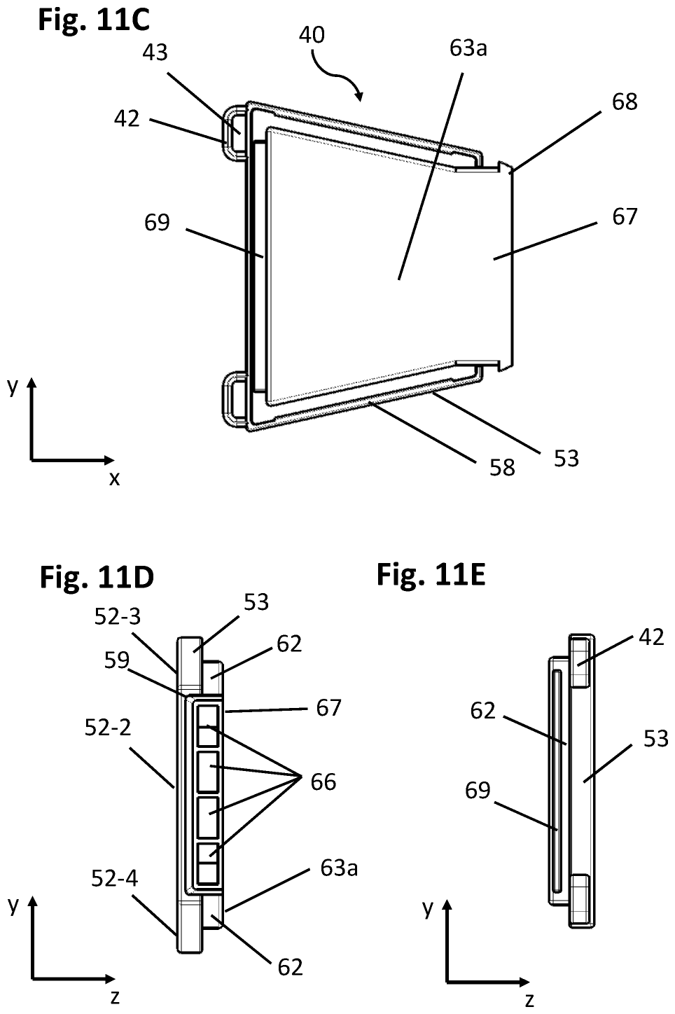

FIGS. 11A-E show a filter element 40 in a usage state from different perspectives. In the usage state, front part 50 and rear part 60 of the filter element 40 are connected to one another and enclose a filter material 70.

It is apparent from FIGS. 11A and 11B that frame 52 and lateral surfaces 53 of the front part 50 enclose the edge 64 of the rear part 60 and cover the support surface 61. In particular, the edge 64 of the rear part 60 is configured and designed to connect front part 50 and rear part 60 to one another. For this purpose, the lateral surface 53 of the front part 50 can be slipped over the edge 64 of the rear part 60 in such a way that lock 58 locks the front part 50 in this position (see also FIG. 11C). It is apparent from FIG. 11D that the recess 59 in the lateral surface 53 enables receiving the tunnel element 67 without influencing the duct 66.

FIG. 12 shows a perspective illustration of a breathing mask 1 with filter elements 40 in cross-section and FIG. 12A shows a detail view from FIG. 12. FIGS. 12/12A show by way of example how the filter element 40 is arranged in the mask wing 14.

The filter element 40 is in a usage state, which means that front part 50 and rear part 60 are connected to one another and enclose a filter material 70. In this case, the edge 64 of the rear part 60 is enclosed by frame 52 and lateral surfaces 53 of the front part 50 in such a way that a firm but generally reversible connection of front part 50 and rear part 60 is provided.

The filter element 40 is received in the receptacle device 16. In this case, the rear wall 63 of the rear part 60 is located adjacent to the receptacle rear wall 18. It is apparent that the receptacle rear wall 18 is connected to the inner wall 15i of the wing duct 15. The material thickness of receptacle rear wall 18 and inner wall 15i can be equal or different. In the specific exemplary embodiment shown, the wall thickness of the receptacle rear wall 18 is made less than the wall thickness of the inner wall 15i. Material can be saved via the lower wall thickness of the receptacle rear wall 18. In some embodiments, the receptacle rear wall 18 can also be absent entirely. The wing duct 15 can be formed stably via the thicker wall thickness of the inner wall 15i and protected from excessive deformations. In some embodiments, the inner wall 15i can include locking recesses 21, in which locking elements of the filter element 40 can engage in order to additionally lock the filter element 40 (not shown).

It is apparent from FIG. 12A that the tunnel element 67, after the filter element 40 is received in the receptacle device 16 of the mask wing 14, is arranged at least in some areas inside at least one partial area of the wing duct 15. In this way, the lumen of the wing duct 15L and the lumen of the tunnel element 67L are fluidically connected to one another.

A (respiratory gas) flow S is schematically indicated by the dashed arrows. The flow S represents the respiratory gas flow of the user after application of the breathing mask 1. The flow S can flow in both directions.

During an inspiration, the respiratory gas flow S can flow through the filter material 70 in the at least one duct 66. The flow S then passes from the duct 66 into the tunnel element 67 and from the tunnel element 67 into the wing duct 15 and from the wing duct 15 in the direction of the mouth and/or knows of the user (not shown). During an expiration, the respiratory gas flow S takes the opposite direction. The flow S can in particular flow through the lumen of the wing duct 15L and the lumen of the tunnel element 67L and the lumen of the duct 66L. From the lumen of the duct 66L, the flow runs through the filter material 70 into the ambient air and vice versa.

In the specific exemplary embodiment shown, the ducts of the tunnel element 67 and the ducts 66 are formed constant in height and width. In some exemplary embodiments, it is conceivable that the duct 67 and/or the duct 66 is formed tapering and/or widening. For example, the duct 66 can be configured and designed in such a way that it widens in a funnel shape in the direction toward the wing duct 15. Such a funnel-shaped widening can positively influence the flow path (not shown).

The specific exemplary embodiment of the filter element 40 according to the drawings includes a locking projection 68 and a locking rail 69. The filter element 40 is locked within the receptacle device 16 or the mask wing 14 via the locking projection 68 and the locking rail 69. Locking projection 68 and locking rail 69 are received for this purpose in each case in corresponding locking recesses 21 of the receptacle side wall 20 or the outer wall 15a of the wing duct 15. Locking recesses 21 in the inner wall 15i and/or the upper wall 15o and/or the lower wall 15u of the wing duct 15 are also conceivable (not shown).

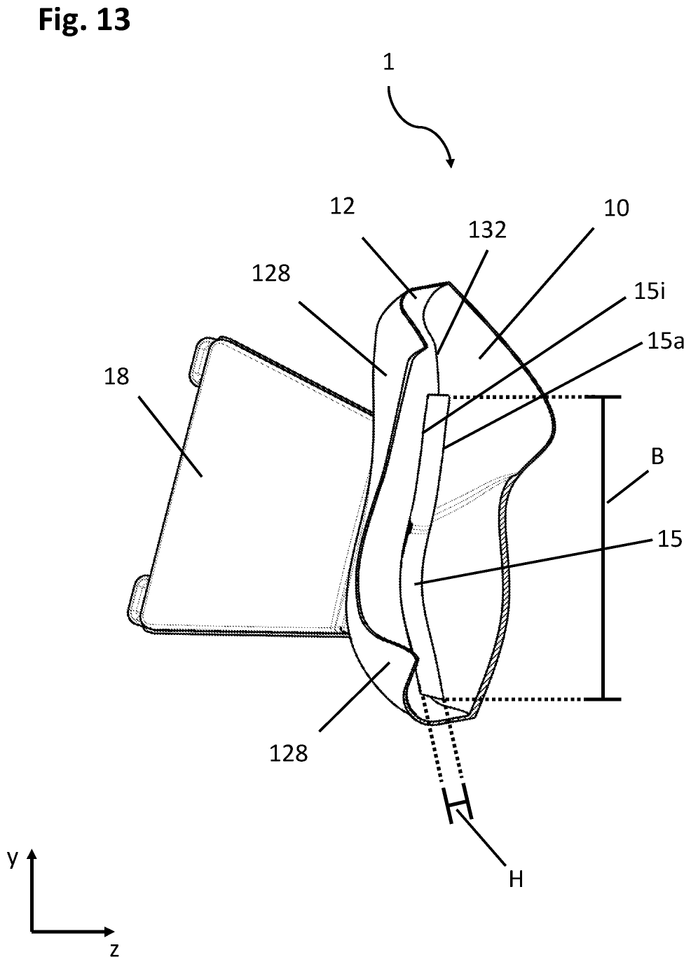

FIG. 13 shows an illustration of a breathing mask 1 in cross-section. The wing duct 15 can have a height H and a width B. The width B of the wing duct 15 is generally greater than the height H of the wing duct 15.

It is apparent from the cross-sectional illustration of FIG. 13 that the wing duct 15 can be arranged in such a way that the wing duct 15 is arranged at least in some sections between mask body 10 and mask bead 12. The wing duct 15 can be arranged, for example, between mask body 10 and mask bead 12 such that the outer wall 15a of the wing duct 15 is connected over the width B to the mask body 10 and the inner wall 15i is connected over the width B to the mask bead 12. In some embodiments, it is conceivable that the wing duct 15 is arranged in or on the mask body 10. In alternative embodiments, it is also conceivable that the wing duct is arranged in or on the mask bead 12 (not shown).

The width B of the wing duct 15 can be in a range between 30 mm and 100 mm, preferably in a range of from about 50 mm to about 80 mm. In a specific exemplary embodiment, the width B of the wing duct 15 is about 72 mm.

The height H of the wing duct 15 can be in a range from about 4 mm to about 12 mm, preferably in a range from about 5 mm to about 8 mm. In a specific exemplary embodiment, the height H of the wing duct 15 is about 6.5 mm.

To sum up, the present invention provides:
1. A breathing mask comprising a mask body, a mask bead, at least one mask wing, and at least one filter element, wherein the mask bead is connected to the mask body, wherein the mask wing is connected to the mask body and/or the mask bead, the mask bead being formed at least in sections for contact on skin of a user, and pressing against the skin of the user during use of the breathing mask in such a way that the breathing mask terminates essentially respiratory gas-tight, wherein the mask bead includes a receptacle opening, which is designed and configured to receive at least nose and mouth of the user during use of the breathing mask, and wherein the filter element is connected to the mask wing, the filter element being configured and designed in such a way that respiratory gas can flow through it at least in some areas.
2. The breathing mask of item 1, wherein the breathing mask comprises at least two mask wings, which are each connected to at least one filter element.
3. The breathing mask of any one of the preceding items, wherein a connection of mask wings and filter elements is reversible or irreversible.
4. The breathing mask of any one of the preceding items, wherein the breathing mask is in a usage state when the filter elements are connected to the mask wings.
5. The breathing mask of any one of the preceding items, wherein the filter element is a filter material and/or comprises a filter material.
6. The breathing mask of any one of the preceding items, wherein the filter material filters and holds back inorganic particles and/or organic particles, in particular germs from the respiratory gas.
7. The breathing mask of any one of the preceding items, wherein the filter material is manufactured from polymer fibers, for example made of polypropylene (PP) and/or polytetrafluoroethylene (PTFE).
8. The breathing mask of any one of the preceding items, wherein the filter material has an area of at least about 5 cm², the area of the filter material being preferably in a range of from about 35 cm² to about 55 cm².
9. The breathing mask of any one of the preceding items, wherein the filter material is exchangeable.
10. The breathing mask of any one of the preceding items, wherein the mask body, the mask bead, and the mask wings with the filter elements, during use of the breathing mask, define a respiratory gas space, in which mouth and nose of the user are located, wherein a respiratory gas flow away from the mouth and nose of the user and toward the mouth and nose of the user is exclusively possible through the filter material.
11. The breathing mask of any one of the preceding items, wherein the respiratory gas space has a volume which is sufficiently small that CO₂ is washed out from the respiratory air of the user.
12. The breathing mask of any one of the preceding items, wherein the respiratory gas space has a volume of at most about 500 ml, preferably at most about 200 ml, for example at most about 120 ml.
13. The breathing mask of any one of the preceding items, wherein the breathing mask is manufactured from an elastomeric and/or a dimensionally stable polymer.
14. The breathing mask of any one of the preceding items, wherein the breathing mask is manufactured from a polymer selected from polycarbonates (PC), polystyrenes (PS), polyamides (PA), polypropylenes (PP), polyoxymethylenes (POM), polyacrylonitriles (PAN), thermoplastic elastomers (TPE), acrylonitrile-styrene acrylates (ASA), acrylonitrile-butadiene-styrenes (ABS), styrene-acrylonitriles (SAN), styrene-methyl-methacrylates (SMMA), polyethylenes (PE), cycloolefin copolymers (COC), polytetrafluorethylenes (PTFE), silicones.
15. The breathing mask of any one of the preceding items, wherein the mask body 10 and/or the mask bead 12 and/or the mask wings 14 are integrally manufactured.
16. The breathing mask of any one of the preceding items, wherein at least the mask body and the mask wings are integrally manufactured and the filter elements are reversibly connected to the mask wings.
17. The breathing mask of any one of the preceding items, wherein the filter elements are manufactured from a dimensionally stable polymer, for example from polypropylene.
18. The breathing mask of any one of the preceding items, wherein the mask body, the mask bead, and the mask wings are essentially manufactured from an elastomeric polymer.
19. The breathing mask of any one of the preceding items, wherein the mask body, the mask bead, and the mask wings are essentially manufactured from a silicone.
20. The breathing mask of any one of the preceding items, wherein the silicone is transparent, having a light transmission of at least about 50%, preferably of at least about 80%, particularly preferably of at least about 90%.
21. The breathing mask of any one of the preceding items, wherein the mask body, the mask bead, and the mask wings have a Shore hardness of from about 30 to about 60 Shore A.
22. The breathing mask of any one of the preceding items, wherein the mask body, the mask bead, and the mask wings have a maximum extensibility of from about 100% to about 800%.
23. The breathing mask of any one of the preceding items, wherein further materials for reinforcing and/or stiffening are introduced and/or applied in and/or on the material of the mask body.
24. The breathing mask of any one of the preceding items, wherein the breathing mask can be disinfected and/or sterilized.
25. The breathing mask of any one of the preceding items, wherein the breathing mask can be autoclaved.
26. The breathing mask of any one of the preceding items, wherein at least the mask body, the mask bead, and the mask wings can be autoclaved.
27. The breathing mask of any one of the preceding items, wherein the breathing mask comprises at least one strap bracket, which is configured to receive at least one head fastening strap.
28. The breathing mask of any one of the preceding items, wherein the strap brackets are formed as projection and contain at least one feedthrough opening for feeding through the head fastening strap.
29. The breathing mask of any one of the preceding items, wherein the strap brackets are arranged on the mask wings and/or on the filter elements.
30. The breathing mask of any one of the preceding items, wherein the strap brackets are arranged on the filter elements.
31. The breathing mask of any one of the preceding items, wherein the mask wings comprise at least one wing duct having a lumen.
32. The breathing mask of any one of the preceding items, wherein the mask wings comprise at least one receptacle device for receiving the filter element, wherein the receptacle device is made substantially complementary in shape to the filter element.
33. The breathing mask of any one of the preceding items, wherein the receptacle device and the filter element essentially have a polygonal basic shape, preferably a square basic shape.
34. The breathing mask of any one of the preceding items, wherein the basic shape of receptacle device and filter element is essentially rhombic or trapezoidal, preferably trapezoidal.
35. The breathing mask of any one of the preceding items, wherein the receptacle device comprises at least one receptacle side wall, which is elastically deformable.
36. The breathing mask of any one of the preceding items, wherein the filter element, after being received in the receptacle device, is at least partially enclosed by the receptacle side wall and is fixed by it in the receptacle device.
37. The breathing mask of any one of the preceding items, wherein the filter element comprises a front part and a rear part and the filter material, wherein the filter material is arranged between the front part and the rear part.
38. The breathing mask of any one of the preceding items, wherein the front part and the rear part are at least partially irreversibly connected to one another or are reversibly connected to one another.
39. The breathing mask of any one of the preceding items, wherein the filter material is arranged exchangeably between the front part and the rear part.
40. The breathing mask of any one of the preceding items, wherein the filter element comprises at least one duct having a lumen.
41. The breathing mask of any one of the preceding items, wherein the rear part comprises a rear wall and a side wall, which delimit the duct at least in some areas.
42. The breathing mask of any one of the preceding items, the rear part comprises at least one duct wall having a base and an end face, wherein the duct wall divides the duct into subunits.
43. The breathing mask of any one of the preceding items, wherein the rear part comprises a support surface, wherein the filter material is placed on the support surface and/or on the end faces of the duct walls in such a way that the duct is delimited at least in some areas by the filter material.
44. The breathing mask of any one of the preceding items, wherein the front part comprises an opening, through which an air exchange takes place between the respiratory gas space and the ambient air.
45. The breathing mask of any one of the preceding items, wherein the filter material is placed between the respiratory gas space and the opening in such a way that the air exchange exclusively takes place through the filter material.
46. The breathing mask of any one of the preceding items, wherein the filter element includes a tunnel element having a lumen.
47. The breathing mask of any one of the preceding items, wherein the rear part comprises the tunnel element.
48. The breathing mask of any one of the preceding items, wherein the tunnel element, in a usage state of the breathing mask, is at least partially arranged in the wing duct of the mask wings.
49. The breathing mask of any one of the preceding items, wherein the tunnel element includes at least one locking projection, via which the filter element is locked in the mask wings.
50. The breathing mask of any one of the preceding items, wherein the rear part and/or the front part includes at least one locking rail, via which the filter element is locked in the mask wings.
51. The breathing mask of any one of the preceding items, wherein the wing duct, the tunnel element, and the duct are connected to one another to conduct gas.
52. The breathing mask of any one of the preceding items, wherein the lumens at least partially define the respiratory gas space, wherein the breathing mask is configured and designed in such a way that a substantially interference-free flow is enabled through the respiratory gas space and the filter material.
53. The breathing mask of any one of the preceding items, wherein the respiratory gas from the mouth and/or nose of the user flows via the lumen 15L into the lumen 67L and from the lumen 67L into the lumen 66L and from the lumen 66L exclusively through the filter material 70 and vice versa.
54. The breathing mask of any one of the preceding items, wherein the wing duct and/or the tunnel element have a hydraulic diameter of at least about 10 mm, preferably at least about 12 mm, particularly preferably at least about 15 mm.
55. A filter element for a breathing mask of any one of the preceding items.

### LIST OF REFERENCE SIGNS

1 breathing mask
2 respiratory gas space
10 mask body
12 mask bead
14 mask wing
15 wing duct
15a outer wall
15i inner wall
15o upper wall
15u lower wall
15L lumen of the wing duct
16 receptacle device
18 receptacle rear wall
20 receptacle side wall
21 locking recesses
24 nose area
28 mouth area
30 attachment area
32 receptacle opening
40 filter element
42 strap bracket
43 feedthrough opening
50 front part
51 opening
52 frame
52-1, 52-2, 52-3, 52-4 sides of the frame
52a outside of the frame
52i inside of the frame
53 lateral surface
54 rib
56 support surface
58 lock
59 recess
60 rear part
61 support surface
61-1, 61-2, 61-3, 61-4 sides of the support surface
62 side wall
63 rear wall
63a rear wall outside
63i rear wall inside
64 edge
65 duct wall
65b base of the duct wall
65s end face of the duct wall
66 duct
66L lumen of the duct
67 tunnel element
67L lumen of the tunnel element
68 locking projection
69 locking rail
70 filter material
90 head
121 corner point
122 corner point
123 corner point
124 corner point
125 nose bridge area
126 lateral area
127 base area
128 support area
129 notch
130 sealing lip
131 edge of the sealing lip
132 transition
A direction toward user
B width
E plane of symmetry
E1 plane
E3 plane
E5 plane
E7 plane
H height
S flow
α angle
β angle

What is claimed is:

1. A breathing mask, wherein the mask comprises a mask body, a mask bead, at least one mask wing, and at least one filter element, the mask bead being connected to the mask body, and the mask wing being connected to the mask body and/or the mask bead, wherein the mask bead is formed at least in sections thereof for contact on skin of a user, and presses against the skin of the user during use of the breathing mask in such a way that the breathing mask terminates essentially respiratory gas-tight, wherein the mask bead includes a receptacle opening, which is designed and configured to receive at least nose and mouth of the user during use of the breathing mask, and wherein the filter element is connected to the mask wing, the filter element being configured and designed in such a way that respiratory gas can flow through it at least in some areas.

2. The breathing mask of claim 1, wherein the breathing mask comprises at least two mask wings, which are each connected to at least one filter element.

3. The breathing mask of claim 1, wherein the filter element is a filter material and/or comprises a filter material.

4. The breathing mask of claim 1, wherein the filter material is manufactured from polymer fibers.

5. The breathing mask of claim 1, wherein the filter material has an area of from 35 cm² to 55 cm².

6. The breathing mask of claim 1, wherein the filter material is exchangeable.

7. The breathing mask of claim 1, wherein the mask body, the mask bead, and the mask wings with the filter elements, during use of the breathing mask, define a respiratory gas space, in which mouth and nose of the user are located, a respiratory gas flow away from the mouth and nose of the user and toward the mouth and nose of the user being exclusively possible through the filter material.

8. The breathing mask of claim 7, wherein the respiratory gas space has a volume of at most 200 ml.

9. The breathing mask of claim 1, wherein at least the mask body and the mask wings are integrally manufactured, and the filter elements are reversibly connected to the mask wings.

10. The breathing mask of claim 1, wherein the mask body, the mask bead, and the mask wings are essentially manufactured from a silicone.

11. The breathing mask of claim 10, wherein the silicone is transparent, having a light transmission of at least 50%.

12. The breathing mask of claim 1, wherein at least the mask body, the mask bead, and the mask wings can be autoclaved.

13. The breathing mask of claim 1, wherein the mask wings comprise at least one wing duct having a lumen.

14. The breathing mask of claim 1, wherein the mask wings comprise at least one receptacle device for receiving the filter element, the receptacle device being made substantially complementary in shape to the filter element.

15. The breathing mask of claim 14, wherein the receptacle device and the filter element essentially have a polygonal basic shape.

16. The breathing mask of claim 14, wherein the receptacle device comprises at least one receptacle side wall, which is elastically deformable.

17. The breathing mask of claim 14, wherein the filter element, after being received in the receptacle device, is at least partially enclosed by a receptacle side wall and is fixed by it in the receptacle device.

18. The breathing mask of claim 1, wherein the filter element comprises a front part and a rear part and the filter material, the filter material being arranged between the front part and the rear part.

19. The breathing mask of claim 1, wherein the filter element comprises at least one duct having a lumen.

20. The breathing mask of claim 1, wherein the filter element comprises a tunnel element having a lumen.
